SEQUENCE LISTING (133 sequences)

SEQ ID NO: 1 - protein, 7 aa, synthetic construct
  REGION 1..7: note = A synthetic sequence
PKKKRKV

SEQ ID NO: 2 - protein, 9 aa, synthetic construct
  REGION 1..9: note = A synthetic sequence
PAAKRVKLD

SEQ ID NO: 3 - skipped

SEQ ID NO: 4 - protein, 16 aa, synthetic construct
  REGION 1..16: note = A synthetic sequence
KRPAATKKAGQAKKKK

SEQ ID NO: 5 - protein, 8 aa, synthetic construct
  REGION 1..8: note = A synthetic sequence
RRRRRRRR

SEQ ID NO: 6 - protein, 15 aa, synthetic construct
  REGION 1..15: note = A synthetic sequence
KWFERWFREWPRKRR

SEQ ID NO: 7 - protein, 9 aa, synthetic construct
  REGION 1..9: note = A synthetic sequence
WWRLSLRWW

SEQ ID NO: 8 - protein, 10 aa, synthetic construct
  REGION 1..10: note = A synthetic sequence
WFRLSLRFWR

SEQ ID NO: 9 - protein, 9 aa, synthetic construct
  REGION 1..9: note = A synthetic sequence
WWRLRSWFR

SEQ ID NO: 10 - protein, 9 aa, synthetic construct
  REGION 1..9: note = A synthetic sequence
WFRLSLRFW

SEQ ID NO: 11 - protein, 18 aa, synthetic construct
  REGION 1..18: note = A synthetic sequence
LFRALWRLLRSLWRLLWK

SEQ ID NO: 12 - protein, 19 aa, synthetic construct
  REGION 1..19: note = A synthetic sequence
LRWWLRWASRWFSRWAWWR

SEQ ID NO: 13 - protein, 4 aa, synthetic construct
  REGION 1..4: note = A synthetic sequence
CADY

SEQ ID NO: 14 - protein, 4 aa, synthetic construct
  REGION 1..4: note = A synthetic sequence
PPTG

SEQ ID NO: 15 - protein, 27 aa, synthetic construct
  REGION 1..27: note = A synthetic sequence
GALFLGFLGAAGSTMGAWSQPKKKRKV

SEQ ID NO: 16 - protein, 21 aa, synthetic construct
  REGION 1..21: note = A synthetic sequence
KETWWETWWTEWSQPKKKRKV

SEQ ID NO: 17 - protein, 21 aa, synthetic construct
  REGION 1..21: note = A synthetic sequence
KETWFETWFTEWSQPKKKRKV

SEQ ID NO: 18 - protein, 21 aa, synthetic construct
  REGION 1..21: note = A synthetic sequence
KETWFETWFTEWSQPKKKRKV

SEQ ID NO: 19 - protein, 20 aa, synthetic construct
  REGION 1..20: note = A synthetic sequence
GLWRALWRLLRSLWRLLWRA

SEQ ID NO: 20 - protein, 22 aa, synthetic construct
  REGION 1..22: note = A synthetic sequence
TPWWRLWTKWHHKRRDLPRKPE

SEQ ID NO: 21 - DNA, 39 bp, synthetic construct
  variation 20: note = A synthetic sequence, n = any nucleotide or an oligonucleotide
  source 1..39: mol_type = other DNA
tgcgtgtgtagtgtgtctgnctcttagggatttgggcgg

SEQ ID NO: 22 - DNA, 27 bp, synthetic construct
  source 1..27: mol_type = other DNA
tcgggatcaacccgagttcacgcaact

SEQ ID NO: 23 - DNA, 25 bp, synthetic construct
  source 1..25: mol_type = other DNA
acactagtacagtcagtacgcacga

SEQ ID NO: 24 - DNA, 25 bp, synthetic construct
  source 1..25: mol_type = other DNA
ttttgcgcctgaagcctccccagga

SEQ ID NO: 25 - DNA, 25 bp, synthetic construct
  source 1..25: mol_type = other DNA
gtccgtatcttggtcgaagatgtac

SEQ ID NO: 26 - DNA, 25 bp, synthetic construct
  source 1..25: mol_type = other DNA
aggttcaatctaccttctgccatgc

SEQ ID NO: 27 - DNA, 25 bp, synthetic construct
  source 1..25: mol_type = other DNA
ctggaattccaaatatgccggcgag

SEQ ID NO: 28 - DNA, 25 bp, synthetic construct
  source 1..25: mol_type = other DNA
atcgttctgggatacaagcttttga

SEQ ID NO: 29 - DNA, 25 bp, synthetic construct
  source 1..25: mol_type = other DNA
aaatatacattattcccatcaaaat

SEQ ID NO: 30 - DNA, 25 bp, synthetic construct
  source 1..25: mol_type = other DNA
ggacaaaaagtctgagtctgacctt

SEQ ID NO: 31 - DNA, 25 bp, synthetic construct
  source 1..25: mol_type = other DNA
gatggcagtatagtcgtcatgagtc

SEQ ID NO: 32 - DNA, 25 bp, synthetic construct
  source 1..25: mol_type = other DNA
tcgggatcaacccgagttcacgcaa

SEQ ID NO: 33 - DNA, 25 bp, synthetic construct
  source 1..25: mol_type = other DNA
actctttctgtcacaagatctgcat

SEQ ID NO: 34 - DNA, 25 bp, synthetic construct
  source 1..25: mol_type = other DNA
aatgtggatggcatagtggcggcgc

SEQ ID NO: 35 - DNA, 25 bp, synthetic construct
  source 1..25: mol_type = other DNA
tacaacctagaggatgagctcacga

SEQ ID NO: 36 - DNA, 25 bp, synthetic construct
  source 1..25: mol_type = other DNA
ttagtgttacatcacatctcgagct

SEQ ID NO: 37 - DNA, 25 bp, synthetic construct
  source 1..25: mol_type = other DNA
taggaggcattcatttttaaggtag

SEQ ID NO: 38 - DNA, 25 bp, synthetic construct
  source 1..25: mol_type = other DNA
gttaccacatcgatctgcgaaaact

SEQ ID NO: 39 - DNA, 25 bp, synthetic construct
  source 1..25: mol_type = other DNA
caccagtggtttgatatacggccta

SEQ ID NO: 40 - DNA, 25 bp, synthetic construct
  source 1..25: mol_type = other DNA
taggttgtggttcccgaatcgtgag

SEQ ID NO: 41 - DNA, 25 bp, synthetic construct
  source 1..25: mol_type = other DNA
gcaacttggcttccgtctaaacaaa

SEQ ID NO: 42 - DNA, 25 bp, synthetic construct
  source 1..25: mol_type = other DNA
gctatccttcttttagcagacagta

SEQ ID NO: 43 - DNA, 25 bp, synthetic construct
  source 1..25: mol_type = other DNA
tactcgtcgcgttttatttttttgc

SEQ ID NO: 44 - DNA, 25 bp, synthetic construct
  source 1..25: mol_type = other DNA
ataagcccccagctactcccgtttt

SEQ ID NO: 45 - DNA, 25 bp, synthetic construct
  source 1..25: mol_type = other DNA
cttcatgtagcaactcattgtgaag

SEQ ID NO: 46 - DNA, 25 bp, synthetic construct
  source 1..25: mol_type = other DNA
tcccaaaatctcgccccccggaata

SEQ ID NO: 47 - DNA, 25 bp, synthetic construct
  source 1..25: mol_type = other DNA
agttgccacaaaaatttactaagtc

SEQ ID NO: 48 - DNA, 25 bp, synthetic construct
  source 1..25: mol_type = other DNA
cagctacattcattatttgtttccg

SEQ ID NO: 49 - DNA, 25 bp, synthetic construct
  source 1..25: mol_type = other DNA
tcatacggaacctcagcccatgaca

SEQ ID NO: 50 - DNA, 25 bp, synthetic construct
  source 1..25: mol_type = other DNA
cacttattagaaatgcatacctata

SEQ ID NO: 51 - DNA, 25 bp, synthetic construct
  source 1..25: mol_type = other DNA
gtctgaaattaattgatcgtcatgc

SEQ ID NO: 52 - DNA, 25 bp, synthetic construct
  source 1..25: mol_type = other DNA
gcatccgattcacacactcgctcac

SEQ ID NO: 53 - DNA, 25 bp, synthetic construct
  source 1..25: mol_type = other DNA
cataacagatgttaaattagcgtaa

SEQ ID NO: 54 - DNA, 25 bp, synthetic construct
  source 1..25: mol_type = other DNA
aaaatcatgagtggtttaccggtag

SEQ ID NO: 55 - DNA, 25 bp, synthetic construct
  source 1..25: mol_type = other DNA
caacccgcaaaaagtctcaggagta

SEQ ID NO: 56 - DNA, 25 bp, synthetic construct
  source 1..25: mol_type = other DNA
cagcttcgccatccctacgggtaag

SEQ ID NO: 57 - DNA, 25 bp, synthetic construct
  source 1..25: mol_type = other DNA
ttctgcatggcgggtatactcacta

SEQ ID NO: 58 - DNA, 25 bp, synthetic construct
  source 1..25: mol_type = other DNA
cccgtagccaaggagcctatacaac

SEQ ID NO: 59 - DNA, 25 bp, synthetic construct
  source 1..25: mol_type = other DNA
tttaagtttccgaatccaacgtaaa

SEQ ID NO: 60 - DNA, 25 bp, synthetic construct
  source 1..25: mol_type = other DNA
ggggagcagcagcgatttgacctag

SEQ ID NO: 61 - DNA, 25 bp, synthetic construct
  source 1..25: mol_type = other DNA
gaaccaaagcgctgcttcccataaa

SEQ ID NO: 62 - DNA, 25 bp, synthetic construct
  source 1..25: mol_type = other DNA
atggttatcttaccctaccaaagga

SEQ ID NO: 63 - DNA, 25 bp, synthetic construct
  source 1..25: mol_type = other DNA
gaccgggttcatttaaccgtacggg

SEQ ID NO: 64 - DNA, 25 bp, synthetic construct
  source 1..25: mol_type = other DNA
ctccaacgcagtagccaggtacaca

SEQ ID NO: 65 - DNA, 25 bp, synthetic construct
  source 1..25: mol_type = other DNA
taactgttgcctctcacatggtcaa

SEQ ID NO: 66 - DNA, 25 bp, synthetic construct
  source 1..25: mol_type = other DNA
cgcttaagtggtatagtctccatcg

SEQ ID NO: 67 - DNA, 25 bp, synthetic construct
  source 1..25: mol_type = other DNA
tcgcaccctatcatagtccgacacc

SEQ ID NO: 68 - DNA, 25 bp, synthetic construct
  source 1..25: mol_type = other DNA
gaatctaaacgttagcaatcggcgt

SEQ ID NO: 69 - DNA, 25 bp, synthetic construct
  source 1..25: mol_type = other DNA
cggttccaggtgggcccgtatacgt

SEQ ID NO: 70 - DNA, 25 bp, synthetic construct
  source 1..25: mol_type = other DNA
agctcccccccgcgttatacctgac

SEQ ID NO: 71 - DNA, 25 bp, synthetic construct
  source 1..25: mol_type = other DNA
gcctagcatccaatcgacgtaccgc

SEQ ID NO: 72 - DNA, 25 bp, synthetic construct
  source 1..25: mol_type = other DNA
cacaacttccatatgtcagttcaac

SEQ ID NO: 73 - DNA, 25 bp, synthetic construct
  source 1..25: mol_type = other DNA
aagcagtctcattcatccatcacta

SEQ ID NO: 74 - DNA, 25 bp, synthetic construct
  source 1..25: mol_type = other DNA
gcttggatttaaaccaagcgtcccg

SEQ ID NO: 75 - DNA, 25 bp, synthetic construct
  source 1..25: mol_type = other DNA
tagatatcgtataatatggggataa

SEQ ID NO: 76 - DNA, 25 bp, synthetic construct
  source 1..25: mol_type = other DNA
taatgccacacgttatgtctcccaa

SEQ ID NO: 77 - DNA, 25 bp, synthetic construct
  source 1..25: mol_type = other DNA
acgaaggcggcatggtaatctgcac

SEQ ID NO: 78 - DNA, 25 bp, synthetic construct
  source 1..25: mol_type = other DNA
ggcgtgggttgaccggatacatgaa

SEQ ID NO: 79 - DNA, 25 bp, synthetic construct
  source 1..25: mol_type = other DNA
tacaagaccgaacctggtttatacc

SEQ ID NO: 80 - DNA, 25 bp, synthetic construct
  source 1..25: mol_type = other DNA
gtggactcgacatccgacagtcagt

SEQ ID NO: 81 - DNA, 25 bp, synthetic construct
  source 1..25: mol_type = other DNA
taaccggttggatagcgattcgatt

SEQ ID NO: 82 - DNA, 25 bp, synthetic construct
  source 1..25: mol_type = other DNA
gccagtctaacagtaattatgcaaa

SEQ ID NO: 83 - DNA, 25 bp, synthetic construct
  source 1..25: mol_type = other DNA
tattcataatacatgagatggcacg

SEQ ID NO: 84 - DNA, 25 bp, synthetic construct
  source 1..25: mol_type = other DNA
cataggattcgtaatgtataagtgt

SEQ ID NO: 85 - DNA, 25 bp, synthetic construct
  source 1..25: mol_type = other DNA
tatctcagaataccgcactcacgtg

SEQ ID NO: 86 - DNA, 25 bp, synthetic construct
  source 1..25: mol_type = other DNA
cggtcaaagcacctggagcgtattc

SEQ ID NO: 87 - DNA, 25 bp, synthetic construct
  source 1..25: mol_type = other DNA
ctgtacttgatccaaggtttgaagg

SEQ ID NO: 88 - DNA, 25 bp, synthetic construct
  source 1..25: mol_type = other DNA
gactacgtcgcttgcaaatcatccg

SEQ ID NO: 89 - DNA, 25 bp, synthetic construct
  source 1..25: mol_type = other DNA
tgtagtctataactccctggcgcag

SEQ ID NO: 90 - DNA, 25 bp, synthetic construct
  source 1..25: mol_type = other DNA
ggtaggcagcacgtttgtgtgaacc

SEQ ID NO: 91 - DNA, 25 bp, synthetic construct
  source 1..25: mol_type = other DNA
ctcttcaccattttatcgccatgca

SEQ ID NO: 92 - DNA, 25 bp, synthetic construct
  source 1..25: mol_type = other DNA
gtctgtctgtatcatccgagcgaca

SEQ ID NO: 93 - DNA, 25 bp, synthetic construct
  source 1..25: mol_type = other DNA
ggaagcgggatatatggtgccgtcc

SEQ ID NO: 94 - DNA, 25 bp, synthetic construct
  source 1..25: mol_type = other DNA
taactcagacagctagctatcgtta

SEQ ID NO: 95 - DNA, 25 bp, synthetic construct
  source 1..25: mol_type = other DNA
taactctgcattgctctcagggagc

SEQ ID NO: 96 - DNA, 25 bp, synthetic construct
  source 1..25: mol_type = other DNA
gtgtcttgtactctgacctgaagcg

SEQ ID NO: 97 - DNA, 25 bp, synthetic construct
  source 1..25: mol_type = other DNA
ggaataagaagtcttagtagcccac

SEQ ID NO: 98 - DNA, 25 bp, synthetic construct
  source 1..25: mol_type = other DNA
aaatagtaaattgaggagccgttta

SEQ ID NO: 99 - DNA, 25 bp, synthetic construct
  source 1..25: mol_type = other DNA
ctacgtataaacggttggttaggtt

SEQ ID NO: 100 - DNA, 25 bp, synthetic construct
  source 1..25: mol_type = other DNA
agcatgaatggaggccgttaacaaa

SEQ ID NO: 101 - DNA, 25 bp, synthetic construct
  source 1..25: mol_type = other DNA
tcccgcagttatcgcggctgtctca

SEQ ID NO: 102 - DNA, 25 bp, synthetic construct
  source 1..25: mol_type = other DNA
cgccgtagggcgtatggcgcgtctg

SEQ ID NO: 103 - DNA, 25 bp, synthetic construct
  source 1..25: mol_type = other DNA
ctacgtgccagtttataccccggaa

SEQ ID NO: 104 - DNA, 25 bp, synthetic construct
  source 1..25: mol_type = other DNA
ccgctagagaaccttgatgattctg

SEQ ID NO: 105 - DNA, 25 bp, synthetic construct
  source 1..25: mol_type = other DNA
tatcttaagtcagtggggctcgtcg

SEQ ID NO: 106 - DNA, 25 bp, synthetic construct
  source 1..25: mol_type = other DNA
tttatgaagagcacatcataagaag

SEQ ID NO: 107 - DNA, 25 bp, synthetic construct
  source 1..25: mol_type = other DNA
tggccgcctagagttaagaactatt

SEQ ID NO: 108 - DNA, 25 bp, synthetic construct
  source 1..25: mol_type = other DNA
gggtcgaatctagttttgtaacagg

SEQ ID NO: 109 - DNA, 25 bp, synthetic construct
  source 1..25: mol_type = other DNA
tgacagtggcgtcacccgttcaccc

SEQ ID NO: 110 - DNA, 25 bp, synthetic construct
  source 1..25: mol_type = other DNA
ctgcgtactggatatgtaaaagatg

SEQ ID NO: 111 - DNA, 25 bp, synthetic construct
  source 1..25: mol_type = other DNA
ggaatgcgtgcagacccgttggttt

SEQ ID NO: 112 - DNA, 25 bp, synthetic construct
  source 1..25: mol_type = other DNA
gtacccaaatgtgagtgacgccatt

SEQ ID NO: 113 - DNA, 25 bp, synthetic construct
  source 1..25: mol_type = other DNA
catctctgtgtacggaaatctttga

SEQ ID NO: 114 - DNA, 25 bp, synthetic construct
  source 1..25: mol_type = other DNA
gtttagcgatccttttgagcattag

SEQ ID NO: 115 - DNA, 25 bp, synthetic construct
  source 1..25: mol_type = other DNA
ctaagttgtaagcgcagcacagcgt

SEQ ID NO: 116 - DNA, 25 bp, synthetic construct
  source 1..25: mol_type = other DNA
gaaacctttgtgcaacgctcgtgtt

SEQ ID NO: 117 - DNA, 25 bp, synthetic construct
  source 1..25: mol_type = other DNA
cttgtctgtcccgctagtttggggg

SEQ ID NO: 118 - DNA, 25 bp, synthetic construct
  source 1..25: mol_type = other DNA
ctaaaaccgggcgtagacgatggtc

SEQ ID NO: 119 - DNA, 25 bp, synthetic construct
  source 1..25: mol_type = other DNA
cggtagtcgccggcttatatgccga

SEQ ID NO: 120 - DNA, 25 bp, synthetic construct
  source 1..25: mol_type = other DNA
ttctgatatctgatgttttatggct

SEQ ID NO: 121 - DNA, 25 bp, synthetic construct
  source 1..25: mol_type = other DNA
agctcgtctagaaaccgtgggccaa

SEQ ID NO: 122 - DNA, 25 bp, synthetic construct
  source 1..25: mol_type = other DNA
gataagggtagagtccagtgaacgg

SEQ ID NO: 123 - DNA, 63 bp, synthetic construct
  source 1..63: mol_type = other DNA
tgcgtgtgtagtgtgtctgtcgggatcaacccgagttcacgcaactcttagggatttgggcgg

SEQ ID NO: 124 - DNA, 63 bp, synthetic construct
  source 1..63: mol_type = other DNA
tgcgtgtgtagtgtgtctggaccgggttcatttaaccgtacgggctcttagggatttgggcgg

SEQ ID NO: 125 - DNA, 63 bp, synthetic construct
  source 1..63: mol_type = other DNA
tgcgtgtgtagtgtgtctggcttggatttaaaccaagcgtcccgctcttagggatttgggcgg

SEQ ID NO: 126 - DNA, 63 bp, synthetic construct
  source 1..63: mol_type = other DNA
tgcgtgtgtagtgtgtctgcggtagtcgccggcttatatgccgactcttagggatttgggcgg

SEQ ID NO: 127 - DNA, 33 bp, synthetic construct
  source 1..33: mol_type = other DNA
tcgtcggcagcgtcagatgtgtataagagacag

SEQ ID NO: 128 - DNA, 34 bp, synthetic construct
  source 1..34: mol_type = other DNA
gtctcgtgggctcggagatgtgtataagagacag

SEQ ID NO: 129 - DNA, 43 bp, synthetic construct
  source 1..43: mol_type = other DNA
aatgatacggcgaccaccgagatctacactcgtcggcagcgtc

SEQ ID NO: 130 - DNA, 39 bp, synthetic construct
  source 1..39: mol_type = other DNA
caagcagaagacggcatacgagatgtctcgtgggctcgg

SEQ ID NO: 131 - DNA, 66 bp, synthetic construct
  variation 45..52: note = A synthetic sequence, n= A, T, G or C
  source 1..66: mol_type = other DNA
ctgtctcttatacacatctccgagcccacgagacnnnnnnnnatctcgtatgccgtcttctgcttg

SEQ ID NO: 132 - DNA, 70 bp, synthetic construct
  variation 44..51: note = A synthetic sequence, n= A, T, G or C
  source 1..70: mol_type = other DNA
ctgtctcttatacacatctgacgctgccgacgannnnnnnngtgtagatctcggtggtcgccgtatcatt

SEQ ID NO: 133 - DNA, 39 bp, synthetic construct; identical to SEQ ID NO: 21
  variation 20: note = A synthetic sequence, n= A, T, G or C
  source 1..39: mol_type = other DNA

(19) United States
(12) Patent Application Publication
(10) Pub. No.: US 2025/0375543 A1
(43) Pub. Date: Dec. 11, 2025

(54) OLIGONUCLEOTIDE-BASED THERAPEUTICS AND USES THEREOF

(71) Applicant: Tod Speer, Willmar, MN (US)

(72) Inventor: Tod Speer, Willmar, MN (US)

(21) Appl. No.: 19/216,566

(22) Filed: May 22, 2025

Related U.S. Application Data
(63) Continuation of application No. 18/251,217, filed on Apr. 28, 2023, now abandoned.
(60) Provisional application No. 63/108,029, filed on Oct. 30, 2020.

(51) Int. Cl. A61K 51/04; A61K 51/08; C07B 59/00

(52) U.S. Cl. CPC A61K 51/0491; A61K 51/0497; A61K 51/088; C07B 59/005

(57) ABSTRACT

Described herein are compounds that are useful for delivering therapeutic, diagnostic, and imaging agents. Also described herein are pharmaceutical compositions containing such compounds and methods of using the compounds and compositions. Also described are processes for manufacture of the compounds and the compositions containing them.

### CROSS-REFERENCE TO RELATED APPLICATIONS

This application is a continuation of and claims the benefit of priority under 35 U.S.C. § 120 to U.S. patent application Ser. No. 18/251,217, filed on Apr. 28, 2023, which is a U.S. National Stage Filing under 35 U.S.C. § 371 from PCT Application No. PCT/US2021/072133, filed Oct. 29, 2021, which claims the benefit of U.S. Provisional Appl. Ser. No. 63/108,029, filed Oct. 30, 2020, each of which is incorporated by reference as if fully set forth herein.

### INCORPORATION BY REFERENCE OF SEQUENCE LISTING

This application contains a Sequence Listing which has been submitted electronically in ST26 format and hereby incorporated by reference in its entirety. Said ST26 file, created on Jun. 23, 2025, is named 5181001US2.xml and is 126,986 bytes in size.

### BACKGROUND

Cancer (malignant neoplasm) is the number two killer of people in the U.S. Each year in the U.S. more than a million people are diagnosed with cancer and half of those will ultimately die from the disease. Cancer occurs when normal living mammalian cells undergo neoplastic (malignant) transformation. Cancer is tenacious in its ability to uncontrollably and rapidly metastasize throughout the mammalian body, thus giving rise to a high mortality rate.

Cancer cure rates have increased dramatically over the years. This positive trend is a result of the widespread use of selective treatment strategies that rely on a variety of targeting structural motifs, including oligonucleotide targeting motifs. Even though compounds containing oligonucleotide targeting motifs may well be selective, assuming one can overcome the obstacle of poor permeability across biologic membranes, they are typically rapidly degraded in vivo by endo- and exo-nucleases.

### BRIEF DESCRIPTION OF THE FIGURES

The drawings illustrate generally, by way of example, but not by way of limitation, various embodiments discussed herein.

FIG. 1 is a photograph of a gel where lane 1 corresponds to a water control; lane 2 corresponds to a doubled stranded DNA (dsDNA) standards ladder; lane 3 corresponds to the PCR product of the compound of formula (IX); and lane 4 corresponds to the PCR product of the compound of formula (X).

FIG. 2 is a photograph of a gel showing the successful isolation of drug candidates.

### SUMMARY

There is therefore a need for selective treatment strategies for cancer that are (i) selective (tumor specific); (ii) overcome the obstacle of poor permeability across biologic membranes; (iii) are substantially stable against endo- and exonucleases without significant alteration of their pharmacokinetics and targeting properties; and (iv) allows for the development of a patient specific theragnostic agent. The compounds disclosed herein include all of these features, such that they are selective for cancer cells, are quickly bound to or are internalized into those cells and are quickly localized into the appropriate compartment(s) in the cell where the compounds can exact their greatest therapeutic effect in a patient specific fashion.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The instant disclosure relates to compounds of the Formula (I):

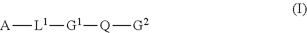

wherein
A comprises at least one avidin-type molecule, each avidin-type molecule comprising one to four monomer units;
L¹ is a linker group;
G¹ is a forward primer binding site;
Q is a randomized single-stranded DNA; and
G² is a reverse primer binding site.

As used herein, the term “avidin-type molecule” includes species that engage in an interaction with biotin with a dissociation constant K_{d} in the order of 10⁻¹⁴ to 10⁻¹⁵ mol/L. Examples of avidin-type molecules include avidin, streptavidin, neutravidin, captavidin, and any other species that engage in an avidin-biotin interaction, such as a nonimmunogenic or low immunogenic forms of streptavidin. See, e.g., Published U.S. Appl. No. 2012/0039879. Typically, but not necessarily, avidin exists as a tetrameric protein, wherein each of the four monomer units that form the tetramer is capable of binding at least one biotin moiety to form what can be termed a “biotin-avidin bond.” As used herein, the term “biotin-avidin bond” and its variants refers to a specific linkage formed between a biotin moiety and an avidin moiety. Typically, a biotin moiety can bind with high affinity to an avidin moiety, with a dissociation constant K_{d} typically in the order of 10⁻¹⁴ to 10⁻¹⁵ mol/L. Typically, such binding occurs via non-covalent interactions. The compounds of the Formula (I) can further include at least one, at least two, at least three or four biotins, or an analog thereof, bound to at least one of the avidin-type molecule.

The disclosure therefore contemplates compounds of the Formula (la):

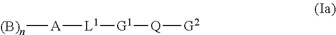

wherein B represents a biotin molecule or an analog thereof and n is an integer from 1 to 4, such that there can be at least one, at least two, at least three or four biotins attached to an avidin-type molecule.

As used herein, the term “biotin” generally refers to a compound comprising a biotin radical of the formula:

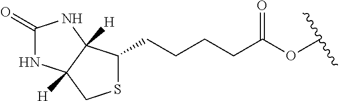

such as, e.g., biotin analogs of the formula:

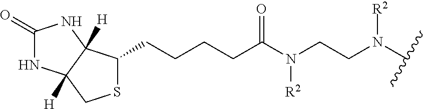

wherein each R² is, independently, H or alkyl. The term “biotin” also includes a biotinidase-resistant biotin analog comprising, e.g., a biotin peptide-bonded to an unnatural D-amino acid that prevents or minimizes recognition by serum biotinidases. See, e.g., Analyt. Biochem. 196:385-89 (1991). The term “biotin” also includes compounds wherein at least one of R² is alkyl, such that the compound is less likely to undergo hydrolysis by a biotinidase, such that the compound as a whole is biotinidase-resistant. In addition, the term “biotin” encompasses labeled biotin compounds of the formula:

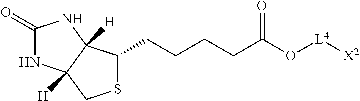

wherein L⁴ is a linker and X² represents a chelating agent, an imaging agent, a diagnostic agent or a therapeutic agent. Suitable imaging agents include, but are not limited to, a radioisotope covalently attached to X², such as ²¹¹At, ¹²⁵I or ¹³¹I, and the like. L⁴ can be any suitable linker and can be the same or different than L¹ and L² as L¹ and L² are defined herein. Thus, for example, L⁴ can be -alkyl-C(O)—NH-alkyl-NH—, such that the compound comprising L⁴-X² is a compound of the formula:

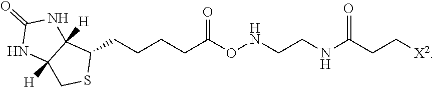

X² can be a chelating agent, an imaging agent, a diagnostic agent or a therapeutic agent, such as a group of the formula:

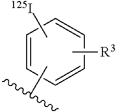

wherein R³ is a substituent as defined herein, such as OH.

The imaging agent or diagnostic agent can be, e.g., a radioactive isotope, such as a radioactive isotope of a metal, coordinated to a chelating group. Illustrative radioactive metal isotopes include technetium, rhenium, gallium, gadolinium, indium, copper, and the like, including isotopes ¹¹¹In, ^{99m}Tc, ⁶⁴Cu, ⁶⁷Cu, ⁶⁷ Ga, ⁶⁸Ga, ¹⁷⁷Lu, ⁸⁹Sr, ¹⁵³Sm, ^{117m}Sn, ²²⁷Th, ²²⁶Th, ²³⁰U, ⁴⁷Sc, ⁸⁶Y or ²²³Ra and the like. Additional illustrative examples of radioactive isotopes include radionuclide imaging agents, such as those described in U.S. Pat. No. 7,128,893, the disclosure of which is incorporated herein by reference.

Illustrative chelating groups include those of the formulae (a)-(l):

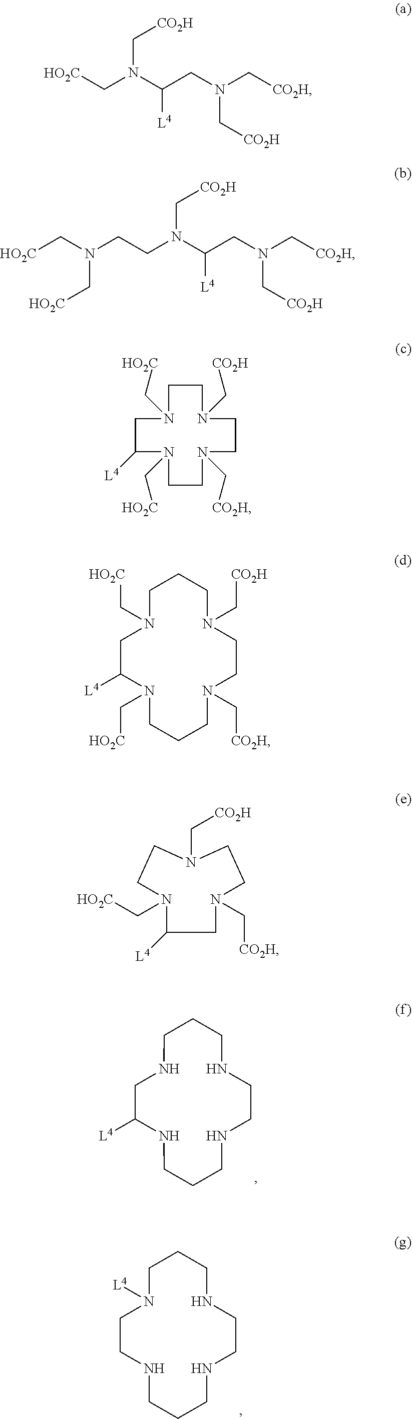

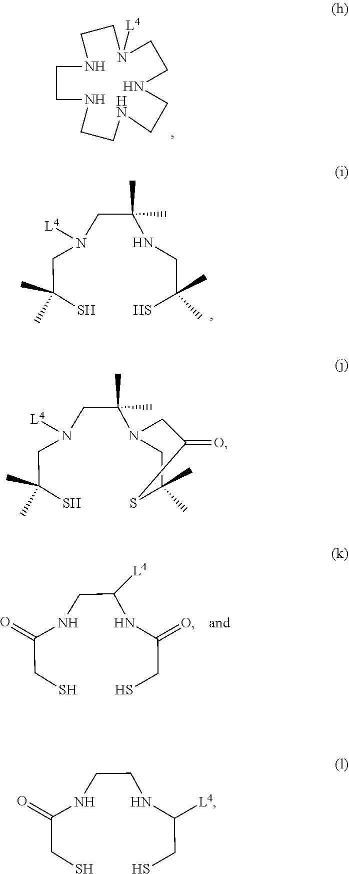

as well as chelating groups disclosed and described in Radiochim Acta 100:653-667 (2012), which is incorporated by reference as if fully set forth herein, wherein L⁴ attaches to the chelating group (a)-(l) on one side and to:

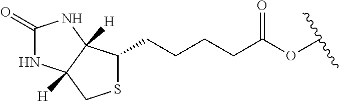 [SMILES: *OC(=O)CCCC[C@@H]1SC[C@@H]2NC(=O)N[C@@H]21]

on the other. The chelating groups (a)-(l) are also known by four-letter abbreviations like EDTA, DTPA, DOTA, TETA, NOTA, Cyclam, CPTA, PCBA, DADT, and MAMA and combinations thereof, each of which can be further substituted. But chelating groups can be attached to L⁴ in any suitable way. For example, chelating groups can be attached to L⁴ via the carboxylic acid groups such as in chelating groups (a′)-(e′):

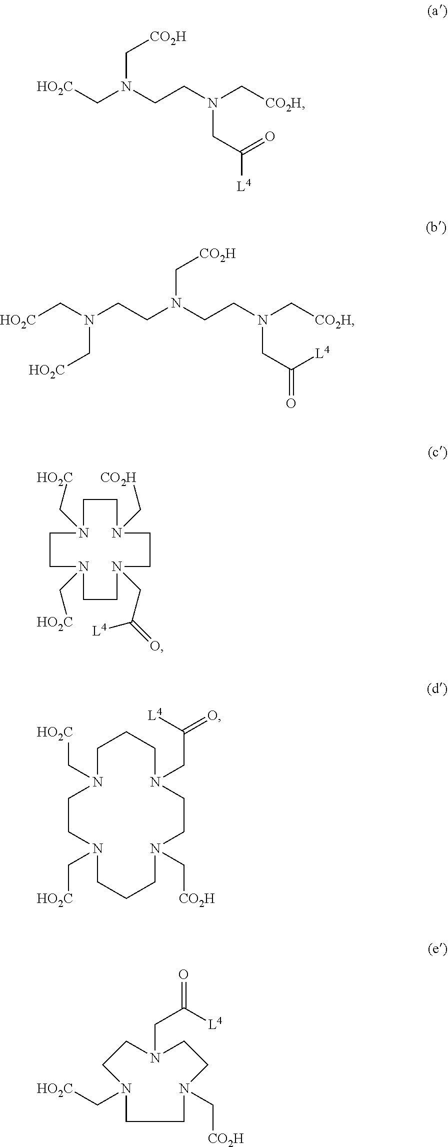

The group X² can also an imaging agent, such as a fluorescent agent. Fluorescent agents include Oregon Green fluorescent agents, including but not limited to Oregon Green 488, Oregon Green 514, and the like, AlexaFluor fluorescent agents, including but not limited to AlexaFluor 488, AlexaFluor 647, and the like, fluorescein, and related analogs, BODIPY fluorescent agents, including but not limited to BODIPY F1, BODIPY 505, and the like, rhodamine fluorescent agents, including but not limited to tetramethylrhodamine, and the like, DyLight fluorescent agents, including but not limited to DyLight 680, DyLight 800, and the like, CW 800, Texas Red, phycoerythrin, and others.

The group X² can also be an imaging agent, such is a PET, a gamma imaging agent or a FRET imaging agent. But the X² is not limited to these three types of imaging agents. PET imaging agents ¹⁸F, ¹¹C, ⁶⁴Cu, ⁶⁵Cu, and the like. FRET imaging agents include ⁶⁴Cu, ⁶⁵Cu, and the like. In the case of ¹⁸F and ¹¹C, the imaging isotope can be present on an aryl groups, such as fluorophenyl, difluorophenyl, fluoronitrophenyl, and the like. The radioisotopes that can be used for imaging may be applied to commercially available dosimetry platforms, such that subsequent therapeutic radioisotope administration can be calculated.

The group X² can be a therapeutic agent, such as radionuclides representing alpha particles, beta particles or Auger electrons, or a chemotherapeutic agent, including the radical of a cytotoxic compound. Cytotoxic compounds include, but are not limited to, compounds that, among other things, enhance tumor permeability, inhibit tumor cell proliferation, promote apoptosis or decrease anti-apoptotic activity in target cells. Example so of cytotoxic compounds include, but are not limited to, aclamycin and aclamycin derivatives, estrogens, selective estrogen receptor modulators (SERMs)), aromatase inhibitors, testosterones (selective androgen receptor modulators (SARMs)), antimetabolites such as cytosine arabinoside, purine analogs, pyrimidine analogs, and methotrexate, busulfan, carboplatin, chlorambucil, cisplatin and other platinum compounds, taxanes, such as tamoxiphen, taxol, paclitaxel, paclitaxel derivatives, TAXOTERE™ and the like, maytansines and analogs and derivatives thereof, cyclophosphamide, daunomycin, doxorubicin, rhizoxin, T2 toxin, plant alkaloids, prednisone, hydroxyurea, teniposide, mitomycins, discodermolides, microtubule inhibitors, epothilones, tubulysin, cyclopropyl benz[e]indolone, seco-cyclopropyl benz[e]indolone, O-Ac-seco-cyclopropyl benz[e]indolone, bleomycin and any other antibiotic, nitrogen mustards, nitrosureas, vincristine, vinblastine, and analogs and derivative thereof such as deacetylvinblastine monohydrazide, colchicine, colchicine derivatives, allocolchicine, thiocolchicine, trityl cysteine, Halicondrin B, dolastatins such as dolastatin 10, amanitins such as α-amanitin, camptothecin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, and any other art-recognized drug or toxin. Other therapeutic agents include penicillins, cephalosporins, vancomycin, erythromycin, clindamycin, rifampin, chloramphenicol, aminoglycoside antibiotics, gentamicin, amphotericin B, acyclovir, trifluridine, ganciclovir, zidovudine, amantadine, ribavirin, maytansines and analogs and derivatives thereof, gemcitabine, and any other art-recognized antimicrobial compound. Also included, but not limited to, are natural dietary products and supplements that behave as chemopreventive agents, such as curcumin, resveratrol, EGCG (epigallocatechin-3-gallate), selenium and emodin.

The term “linker,” which is used interchangeably with “linker group” herein (e.g., linker groups L¹, L², L³, L⁴, L⁵), can be any suitable linker (e.g., divalent linkers and polyvalent linkers). For example, the linker can be a hydrophilic linker, such as a linker that comprises one or more of an amino acid (which can be the same or different), a polyethylene glycol (PEG) monomer, a PEG oligomer, a PEG polymer, or a combination of an any of the foregoing. The linker can comprise an oligomer of peptidoglycans, glycans, or anions.

The linker groups (e.g., L¹, L², L³, L⁴, and L⁵) described herein can have any suitable length and chemical composition. For example, the linker can have a chain length of at least about 7 atoms in length. In one variation, the linker is at least about 10 atoms in length. In one variation, the linker is at least about 14 atoms in length. In another variation, the linker is between about 7 and about 31 (such as, about 7 to 31, 7 to about 31 or 7 to 31) between about 7 and about 24 (such as, about 7 to 24, 7 to about 24 or 7 to 24), or between about 7 and about 20 (such as, about 7 to 20, 7 to about 20 or 7 to 20) atoms in length. In another variation, the linker is between about 14 and about 31 (such as, about 14 to 31, 14 to about 31 or 14 to 31), between about 14 and about 24 (such as, about 14 to 24, 14 to about 24 or 14 to 24), or between about 14 and about 20 (such as, about 14 to 20, 14 to about 20 or 14 to 20) atoms in length. In another variation, the linker can have a chain length of at least 7 atoms, at least 14 atoms, at least 20 atoms, at least 25 atoms, at least 30 atoms, at least 40 atoms; or from 1 to 15 atoms, 1 to 5 atoms, 5 to 10 atoms, 5 to 20 atoms, 10 to 40 atoms or 25 to 100 atoms. An example of a linker group having a chain length of 1 to 5 atoms is a group of the formula:

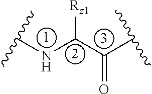

wherein R_{z1} can be H, alkyl, arylalkyl, or -alkyl-S-alkyl or the side-chain of any naturally- or non-naturally occurring amino acid, and the like; and the numbers represent the atoms that are counted as being part of the chain, which in this case is 3 atoms. Examples of R_{z1} include H (i.e., side chain of glycine), alkyl (e.g., side chain of alanine, valine, isoleucine, and leucine), -alkyl-S-alkyl (e.g., side chain of methionine), arylalkyl (e.g., side chain of phenylalanine, tyrosine, and tryptophan), and the like. The atom to which R_{z1} is attached can be chiral and can have any suitable relative configuration, such as a D- or L-configuration.

The atoms used in forming the linker can be combined in all chemically relevant ways, such as chains of carbon atoms forming alkylene groups, chains of carbon and oxygen atoms forming polyoxyalkylene groups, chains of carbon and nitrogen atoms forming polyamines, and others. In addition, it is to be understood that the bonds connecting atoms in the chain may be either saturated or unsaturated, such that for example, alkanes, alkenes, alkynes, cycloalkanes, arylenes, imides, and the like may be divalent radicals that are included in L. In addition, it is to be understood that the atoms forming the linker may also be cyclized upon each other to form saturated or unsaturated divalent cyclic radicals in the linker, such as radicals of the formulae:

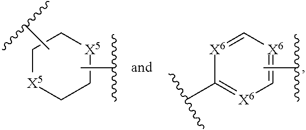

wherein each X⁵ is independently CH₂, N (when there is a bond attached to X⁵), NH or O and each X⁶ is independently N, C (when there is a bond attached to X⁶) or CH. Thus, for example, the foregoing groups can be of the formulae:

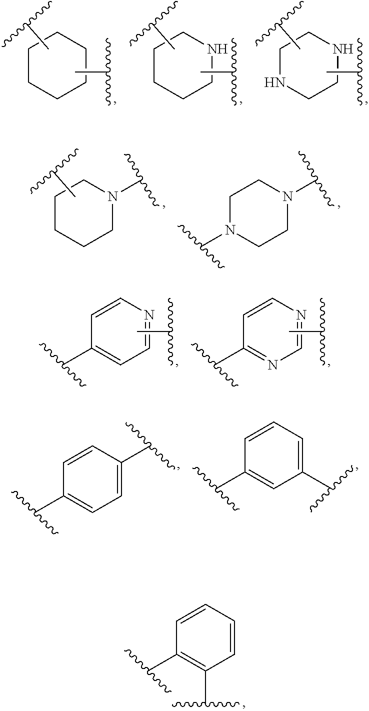

and the like. In each of the foregoing and other the linker groups described herein, the chain forming the linker may be substituted or unsubstituted.

Alternatively, or in addition to chain length, the linker can have any suitable substituents that can affect the hydrophobicity or hydrophilicity of L. Thus, for example, the linker can have a hydrophobic side chain group, such as an alkyl, cycloalkyl, aryl, arylalkyl, or like group, each of which is optionally substituted. If the linker were to include one or more amino acids, the linker can contain hydrophobic amino acid side chains, such as one or more amino acid side chains from phenylalanine (Phe) and tyrosine (Tyr), including substituted variants thereof, and analogs and derivatives of such side chains. Variants, analogs, and derivatives of these side chains include, for example, groups such as:

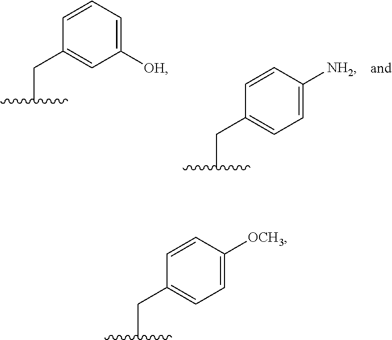 [SMILES: *Cc1cccc(O)c1 ; *Cc1ccc(N)cc1 ; *Cc1ccc(OC)cc1]

which are respectively a variant of tyrosine, an amine analog of tyrosine, and a methoxy derivative of tyrosine. Other variants, analogs, and derivatives are contemplated.

The linker can comprise portions that are neutral under physiological conditions. But the linker can comprise portions that can be protonated or deprotonated to carry one or more positive or one or more negative charges, respectively. Or the linker can comprise neutral portions and portions that may be protonated to carry one or more positive charges. Examples of neutral portions include poly hydroxyl groups, such as sugars, carbohydrates, saccharides, inositols, and the like, and/or polyether groups, such as polyoxyalkylene groups including polyoxyethylene, polyoxypropylene, and the like. Examples of portions that can be protonated to carry one or more positive charges include amino groups, such as polyaminoalkylenes including ethylene diamines, propylene diamines, butylene diamines and the like, and/or heterocycles including pyrrolidines, piperidines, piperazines, and other amino groups, each of which can be optionally substituted. Examples of portions that can be deprotonated to carry one or more negative charges include carboxylic acids, such as aspartic acid, glutamic acid, and longer chain carboxylic acid groups, and sulfuric acid esters, such as alkyl esters of sulfuric acid.

Illustrative polyoxyalkylene groups include those of a specific length range from about 4 to about 20 polyoxyalkylene (e.g., polyethylene glycol) groups, such as about 4 to 20, 4 to about 20 or 4 to 20 polyoxyalkylene groups. Illustrative alkyl sulfuric acid esters may also be introduced with click chemistry directly into the backbone. Illustrative linker groups comprising polyamines include linker groups comprising EDTA and DTPA radicals:

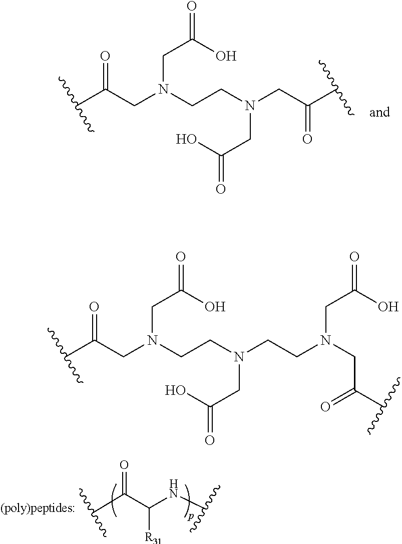

β-amino acids, and the like:

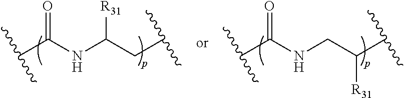

and combinations thereof, wherein each R₃₁ is independently H, alkyl, arylalkyl, heterocyclylalkyl, ureido, aminoalkyl, alkylthio or amidoalkyl, such as in the side chains of naturally-occurring amino acids like alanine, valine, leucine, isoleucine, phenylalanine, tyrosine, tryptophan, serine, threonine, asparagine, methionine, lysine, arginine, and histidine. Non-naturally occurring amino acids are also contemplated herein.

As discussed herein, the linker can include at least one releasable portion. In one variation, the linker includes at least two releasable linkers (e.g., cleavable linkers). The choice of a releasable linker or a non-releasable linker can be made independently for each application or configuration of the compounds described herein. The releasable linkers described herein comprise various atoms, chains of atoms, functional groups, and combinations of functional groups. For example, the releasable linker can comprise about 1 to about 30 atoms (e.g., about 1 to 30, 1 to about 30, and 1 to 30 atoms), or about 2 to about 20 atoms (e.g., about 2 to 20, 2 to about 20, and 2 to 20 atoms). Lower molecular weight linkers (e.g., those having an approximate molecular weight of about 30 g/mol to about 1,000 g/mol, such as from about 30 g/mol to about 300 g/mol, about 100 g/mol to about 500 g/mol or about 150 g/mol to about 600 g/mol) are also described. Precursors to such linkers may be selected to have either nucleophilic or electrophilic functional groups, or both, optionally in a protected form with a readily cleavable protecting group to facilitate their use in synthesis of the intermediate species.

The term “releasable linker” as used herein refers to a linker that includes at least one bond that can be broken under physiological conditions (e.g., a pH-labile, acid-labile, oxidatively-labile, or enzyme-labile bond). Releasable groups also include photochemically-cleavable groups. Examples of photochemically-cleavable groups include 2-(2-nitrophenyl)-ethan-2-ol groups and linkers containing o-nitrobenzyl, desyl, trans-o-cinnamoyl, m-nitrophenyl or benzylsulfonyl groups (see, for example, Dorman and Prestwich, Trends Biotech. 18:64-77 (2000); and Greene and Wuts, Protective Groups in Organic Synthesis, 2nd ed., John Wiley & Sons, New York (1991)).

The cleavable bond or bonds may be present in the interior of a cleavable linker and/or at one or both ends of a cleavable linker. It should be appreciated that such physiological conditions resulting in bond breaking include standard chemical hydrolysis reactions that occur, for example, at physiological pH, or as a result of compartmentalization into a cellular organelle such as an endosome having a lower pH than cytosolic pH. Illustratively, the bivalent linkers described herein can undergo cleavage under other physiological or metabolic conditions, such as by the action of a glutathione mediated mechanism. It is appreciated that the lability of the cleavable bond may be adjusted by including functional groups or fragments within the bivalent linker that are able to assist or facilitate such bond breakage, also termed anchimeric assistance. The lability of the cleavable bond can also be adjusted by, for example, substitutional changes at or near the cleavable bond, such as including alpha branching adjacent to a cleavable disulfide bond, increasing the hydrophobicity of substituents on silicon in a moiety having a silicon-oxygen bond that may be hydrolyzed, homologating alkoxy groups that form part of a ketal or acetal that may be hydrolyzed, and the like. In addition, it is appreciated that additional functional groups or fragments may be included within the bivalent linker that are able to assist or facilitate additional fragmentation of the conjugates after bond breaking of the releasable linker, when present.

In one example, the linker can comprise one or more releasable linkers that cleave under the conditions described herein by a chemical mechanism involving beta elimination. Such releasable linkers include beta-thio, beta-hydroxy, and beta-amino substituted carboxylic acids and derivatives thereof, such as esters, amides, carbonates, carbamates, and ureas. Such linkers also include 2- and 4-thioarylesters, carbamates, and carbonates.

An example of a releasable linker includes a linker of the formula:

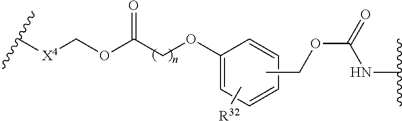

wherein X⁴ is NR, n is an integer selected from 0, 1, 2, and 3, R32 is hydrogen, or a substituent, including a substituent capable of stabilizing a positive charge inductively or by resonance on the aryl ring, such as alkoxy, and the like. The releasable linker can be further substituted.

Assisted cleavage of releasable portions of the linker can include mechanisms involving benzylium intermediates, benzyne intermediates, lactone cyclization, oxonium intermediates, beta-elimination, and the like. In addition to fragmentation subsequent to cleavage of a releasable portion of L, the initial cleavage of the releasable linker may be facilitated by an anchimerically assisted mechanism. Thus, in the example of a releasable portion of the linker given above, the hydroxyalkanoic acid, which may cyclize, facilitates cleavage of the methylene bridge, by for example an oxonium ion, and facilitates bond cleavage or subsequent fragmentation after bond cleavage of the releasable linker. Alternatively, acid catalyzed oxonium ion-assisted cleavage of the methylene bridge may begin a cascade of fragmentation of this illustrative bivalent linker, or fragment thereof. Alternatively, acid-catalyzed hydrolysis of the carbamate may facilitate the beta elimination of the hydroxyalkanoic acid, which may cyclize, and facilitate cleavage of methylene bridge, by for example an oxonium ion. It is appreciated that other chemical mechanisms of bond breakage or cleavage under the metabolic, physiological, or cellular conditions described herein may initiate such a cascade of fragmentation. It is appreciated that other chemical mechanisms of bond breakage or cleavage under the metabolic, physiological, or cellular conditions described herein can initiate such a cascade of fragmentation.

Illustrative mechanisms for cleavage of the bivalent linkers described herein include the following 1,4 and 1,6 fragmentation mechanisms for carbonates and carbamates:

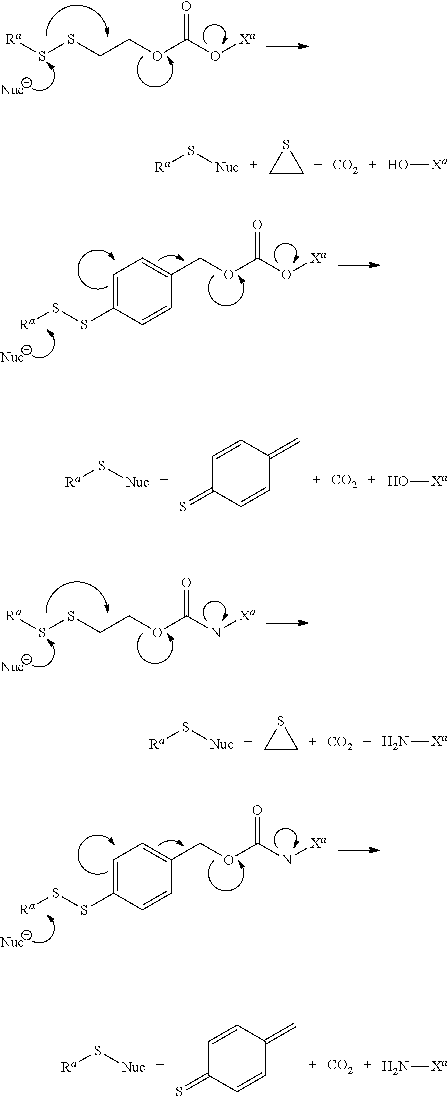

wherein Nuc⁻ is an exogenous or endogenous nucleophile, glutathione, or bioreducing agent, and the like, and R^{a} and X^{a} are connected through other portions of the bivalent linker. The location of R^{a} and X^{a} can be switched such that, e.g., the resulting products are X^{a}—S-Nuc and HO—R^{a} H₂N—R^{a}.

Although the above fragmentation mechanisms are depicted as concerted mechanisms, any number of discrete steps can take place to effect the ultimate fragmentation of the bivalent linker to the final products shown. For example, the bond cleavage can also occur by acid catalyzed elimination of the carbamate moiety, which may be anchimerically assisted by the stabilization provided by either the aryl group of the beta sulfur or disulfide illustrated in the above examples. In those variations of this embodiment, the releasable linker is the carbamate moiety. Alternatively, the fragmentation can be initiated by a nucleophilic attack on the disulfide group, causing cleavage to form a thiolate. The thiolate can intermolecularly displace a carbonic acid or carbamic acid moiety and form the corresponding thiacyclopropane. In the case of the benzyl-containing bivalent linkers, following an illustrative breaking of the disulfide bond, the resulting phenyl thiolate can further fragment to release a carbonic acid or carbamic acid moiety by forming a resonance-stabilized intermediate. In any of these cases, the releasable nature of the illustrative bivalent linkers described herein may be realized by whatever mechanism may be relevant to the chemical, metabolic, physiological, or biological conditions present.

As described above, therefore, releasable linkers can comprise a disulfide group. Further examples of releasable linkers comprised in the linker can include divalent radicals comprising alkyleneaziridin-1-yl, alkylenecarbonylaziridin-1-yl, carbonylalkylaziridin-1-yl, alkylenesulfoxylaziridin-1-yl, sulfoxylalkylaziridin-1-yl, sulfonylalkylaziridin-1-yl, or alkylenesulfonylaziridin-1-yl groups, wherein each of the releasable linkers is optionally substituted. Additional examples of releasable linkers comprised in the linker can include divalent radicals comprising methylene, 1-alkoxyalkylene, 1-alkoxycycloalkylene, 1-alkoxyalkylenecarbonyl, 1-alkoxycycloalkylenecarbonyl, carbonylarylcarbonyl, carbonyl(carboxyaryl)carbonyl, carbonyl(biscarboxyaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio or carbonylalkylthio groups, wherein each of the releasable linkers is optionally substituted.

Additional examples of releasable linkers comprised in the linker can include an oxygen atom and methylene, 1-alkoxyalkylene, 1-alkoxycycloalkylene, 1-alkoxyalkylenecarbonyl or 1-alkoxycycloalkylenecarbonyl groups, wherein each of the releasable linkers is optionally substituted. Alternatively, the releasable linker includes an oxygen atom and a methylene group, wherein the methylene group is substituted with an optionally substituted aryl, and the releasable linker is bonded to the oxygen to form an acetal or ketal. Further, the releasable linker can include an oxygen atom and a sulfonylalkyl group, and the releasable linker is bonded to the oxygen to form an alkylsulfonate.

Additional examples of releasable linkers comprised in the linker can include a nitrogen and iminoalkylidenyl, carbonylalkylideniminyl, iminocycloalkylidenyl, and carbonylcycloalkylideniminyl groups, wherein each of the releasable linkers is optionally substituted and the releasable linker is bonded to the nitrogen to form an hydrazone. In an alternate configuration, the hydrazone can be acylated with a carboxylic acid derivative, an orthoformate derivative, or a carbamoyl derivative to form various acylhydrazone releasable linkers.

Additional examples of releasable linkers comprised in the linker can include an oxygen atom and alkylene(dialkylsilyl), alkylene(alkylarylsilyl), alkylene(diarylsilyl), (dialkylsilyl)aryl, (alkylarylsilyl)aryl or (diarylsilyl)aryl groups, wherein each of the releasable linkers is optionally substituted and the releasable linker is bonded to the oxygen to form a silanol.

Additional examples of releasable linkers comprised in the linker can include two independent nitrogens and carbonylarylcarbonyl, carbonyl(carboxyaryl)carbonyl, or carbonyl(biscarboxyaryl)carbonyl. The releasable linker can be bonded to the heteroatom nitrogen to form an amide, and also bonded to X^{a} or R^{a} via an amide bond.

Additional examples of releasable linkers comprised in the linker can include an oxygen atom, a nitrogen, and a carbonylarylcarbonyl, carbonyl(carboxyaryl)carbonyl, or carbonyl(biscarboxyaryl)carbonyl. The releasable linker can form an amide, and also can be bonded to X^{a} or R^{a} via an amide bond.

The linker can comprise an optionally substituted 1-alkylenesuccinimid-3-yl group and a releasable portion comprising methylene, 1-alkoxyalkylene, 1-alkoxycycloalkylene, 1-alkoxyalkylenecarbonyl or 1-alkoxycycloalkylenecarbonyl groups, each of which can be optionally substituted, to form a succinimid-1-ylalkyl acetal or ketal.

The linker can comprise carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl or 1-(carbonyltetrahydrofuranyl)succinimid-3-yl, each of which is optionally substituted. In this example, the linker can further comprise an additional nitrogen such that the linker comprises alkylenecarbonyl, cycloalkylenecarbonyl, carbonylalkylcarbonyl or 1-(carbonylalkyl)succinimid-3-yl groups, each of which is optionally substituted, bonded to the nitrogen to form an amide. Alternatively, the linker can further comprise a sulfur atom and alkylene or cycloalkylene groups, each of which is optionally substituted with carboxy, and is bonded to the sulfur to form a thiol. In yet another example, the linker comprises a sulfur atom and 1-alkylenesuccinimid-3-yl and 1-(carbonylalkyl)succinimid-3-yl groups bonded to the sulfur to form a succinimid-3-ylthiol.

The linker can comprise a nitrogen and a releasable portion comprising alkyleneaziridin-1-yl, carbonylalkylaziridin-1-yl, sulfoxylalkylaziridin-1-yl, or sulfonylalkylaziridin-1-yl, each of which is optionally substituted. In this example, the linker can comprise carbonyl, thionocarbonyl, alkylenecarbonyl, cycloalkylenecarbonyl, carbonylalkylcarbonyl, or 1-(carbonylalkyl)succinimid-3-yl, each of which is optionally substituted, and bonded to the releasable portion to form an aziridine amide.

Examples of the linker include alkylene-amino-alkylenecarbonyl, alkylene-thio-(carbonylalkylsuccinimid-3-yl), and the like, as further illustrated by the following formulae:

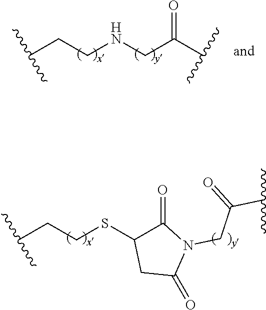

wherein x′ and y′ are each independently 1, 2, 3, 4, or 5.

The linker can have any suitable assortment of atoms in the chain, including C (e.g., —CH₂—, C(O)), N (e.g., NH, NR^{b}, wherein R^{b} is, e.g., H, alkyl, alkylaryl, and the like), O (e.g., —O—), P (e.g., —O—P(O)(OH)O—), and S (e.g., —S—). For example, the atoms used in forming the linker may be combined in all chemically relevant ways, such as chains of carbon atoms forming alkyl groups, chains of carbon and oxygen atoms forming polyoxyalkyl groups, chains of carbon and nitrogen atoms forming polyamines, and others, including rings, such as those that form aryl and heterocyclyl groups (e.g., triazoles, oxazoles, and the like). In addition, the bonds connecting atoms in the chain in the linker may be either saturated or unsaturated, such that for example, alkanes, alkenes, alkynes, cycloalkanes, arylenes, imides, and the like may be divalent radicals that are included in L. Further, the chain-forming the linker can be substituted or unsubstituted.

Additional examples of linker groups include the groups 1-alkylsuccinimid-3-yl, carbonyl, thionocarbonyl, alkyl, cycloalkyl, alkylcycloalkyl, alkylcarbonyl, cycloalkylcarbonyl, carbonylalkylcarbonyl, 1-alkylsuccinimid-3-yl, 1-(carbonylalkyl) succinimid-3-yl, alkylsulfoxyl, sulfonylalkyl, alkylsulfoxylalkyl, alkylsulfonylalkyl, carbonyltetrahydro-2H-pyranyl, carbonyltetrahydrofuranyl, 1-(carbonyltetrahydro-2H-pyranyl) succinimid-3-yl, and 1-(carbonyltetrahydrofuranyl) succinimid-3-yl, wherein each group can be substituted or unsubstituted. Any of the aforementioned groups can be a linker or can be included as a portion of L. In some instances, any of the aforementioned groups can be used in combination (or more than once) (e.g., -alkyl-C(O)-alkyl) and can further comprise an additional nitrogen (e.g., alkyl-C(O)—NH—, —NH-alkyl-C(O)— or —NH-alkyl-), oxygen (e.g., -alkyl-O-alkyl-) or sulfur (e.g., -alkyl-S-alkyl-). Examples of such linker groups are alkylcarbonyl, cycloalkylcarbonyl, carbonylalkylcarbonyl, 1-(carbonylalkyl)succinimid-3-yl, and succinimid-3-ylthiol, wherein each group can be substituted or unsubstituted.

In some instances, the linker can be formed via click chemistry/click chemistry-derived. Those of skill in the art understand that the terms “click chemistry” and “click chemistry-derived” generally refer to a class of small molecule reactions commonly used in conjugation, allowing the joining of substrates of choice with specific molecules. Click chemistry is not a single specific reaction, but describes a way of generating products that follow examples in nature, which also generates substances by joining small modular units. In many applications, click reactions join a biomolecule and a reporter molecule. Click chemistry is not limited to biological conditions: the concept of a “click” reaction has been used in pharmacological and various biomimetic applications. However, they have been made notably useful in the detection, localization and qualification of biomolecules.

Click reactions can occur in one pot, typically are not disturbed by water, can generate minimal byproducts, and are “spring-loaded”—characterized by a high thermodynamic driving force that drives it quickly and irreversibly to high yield of a single reaction product, with high reaction specificity (in some cases, with both regio- and stereo-specificity). These qualities make click reactions suitable to the problem of isolating and targeting molecules in complex biological environments. In such environments, products accordingly need to be physiologically stable and any byproducts need to be non-toxic (for in vivo systems).

Click chemistry examples include examples where the linker can be derived from copper-catalyzed azide-alkyne cycloaddition (CuAAC), strain-promoted azide-alkyne cycloaddition (SPAAC), inverse electron demand Diels-Alder reaction (IEDDA), and Staudinger ligation (SL). For example, X^{a} and R^{a} can be linked to each other as shown in Schemes 1-5:

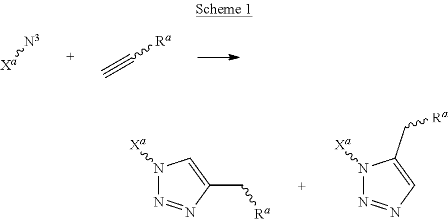

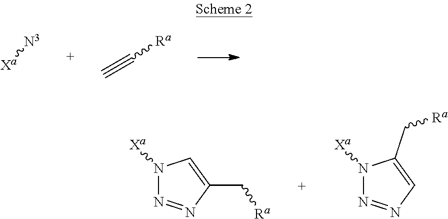

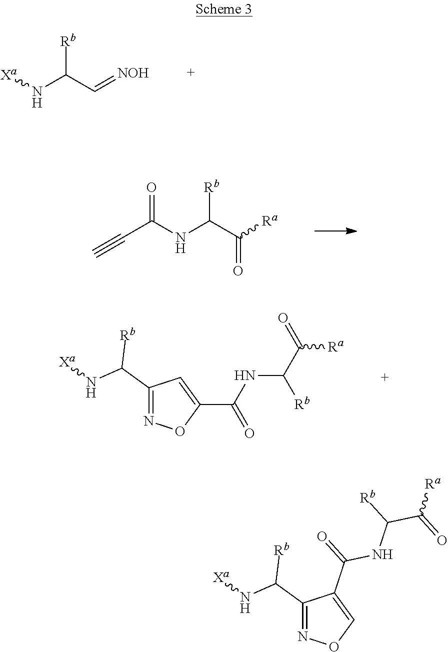

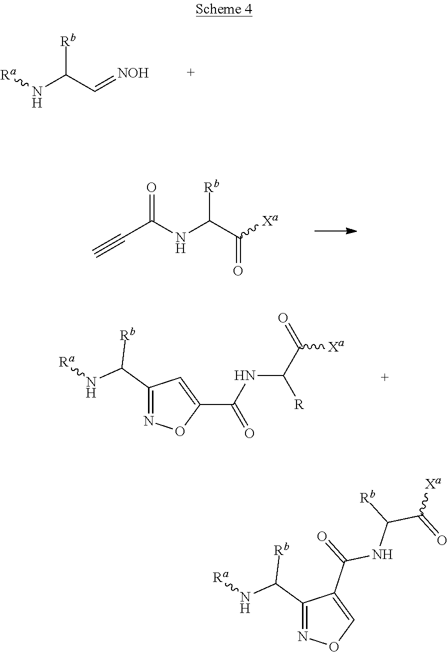

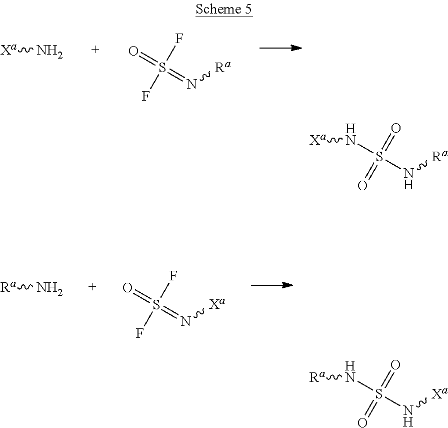

wherein each R^{b} is independently H, alkyl, arylalkyl, -alkyl-S-alkyl or arylalkyl or the side-chain of any naturally- or non-naturally occurring amino acid and the like. In Schemes 1-5, the wavy line connected to X^{a} and R^{a} represents a linkage between X^{a} and R^{a} and the groups to which they are attached. It should be appreciated that in Schemes 1-5, the triazole, oxazole, and the —NH—SO₂—NH— group would be considered to be part of the linker.

The linker can be a linker selected from the group consisting of pegylated-, alkyl-, sugar-, and peptide-based dual linker; the linker is either a non-releasable linker or a releasable linker bivalently covalently attached to e.g., any one of the groups X², T, and G¹, described herein.

For example, the linker can comprise one or more of the groups:

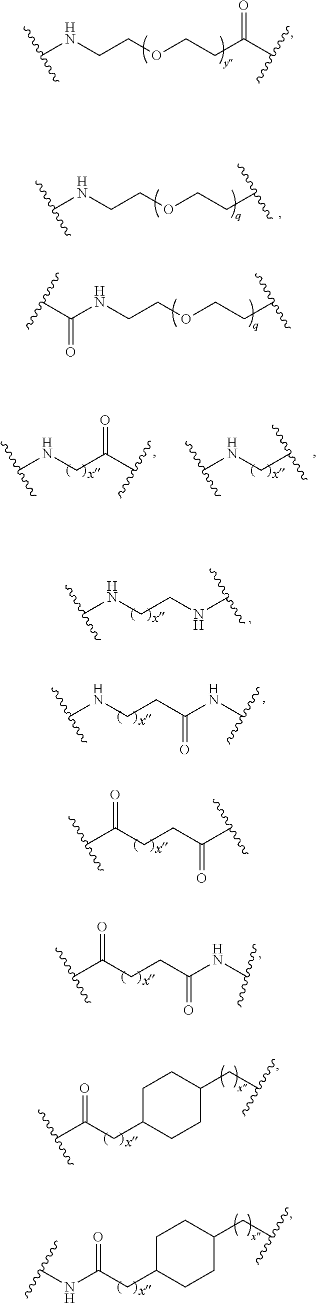

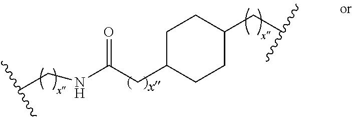

wherein
q is an integer that encompasses a polyethylene (PEG) group having an average Mn of 5,000;
each x″ independently is an integer from 0 to 10, and
each y″ independently is an integer from 3 to 100.

In some aspects, x″ is an integer from 3 to 10.

The linker can be:

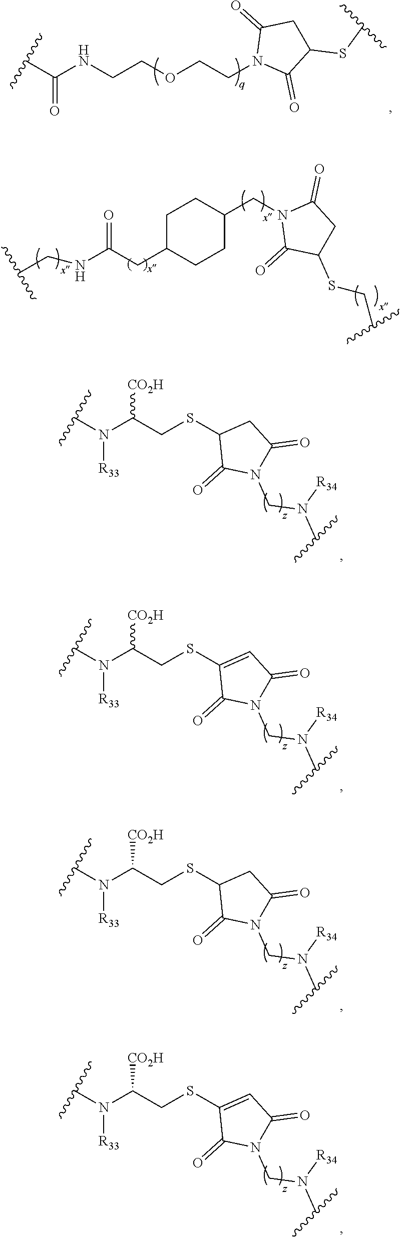

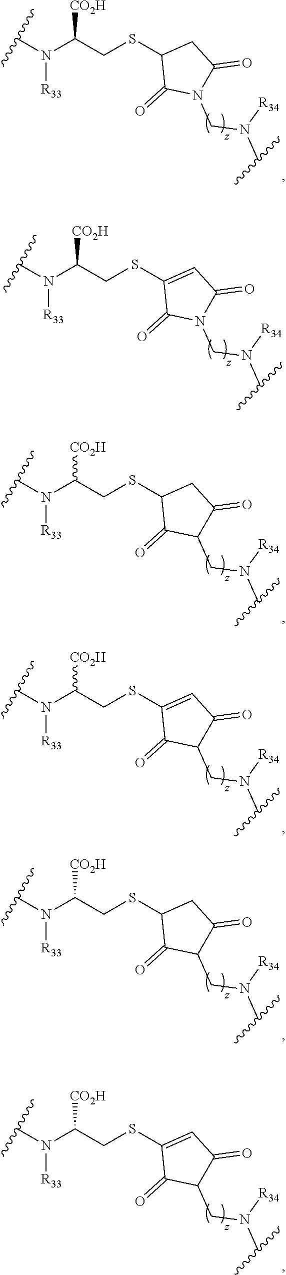

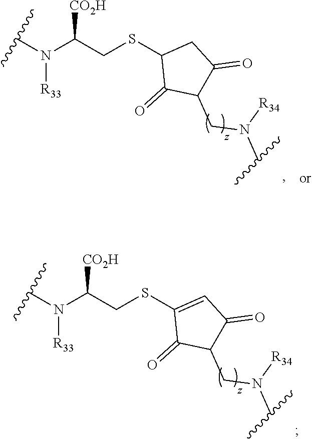

wherein each of R₃₃ and R₃₄ is independently H or C₁-C₆ alkyl;
and z is an integer from 1 to 8.

For example, the linker can be

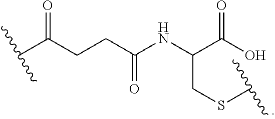 [SMILES: *SCC(NC(=O)CCC(*)=O)C(=O)O]

The linker can be:

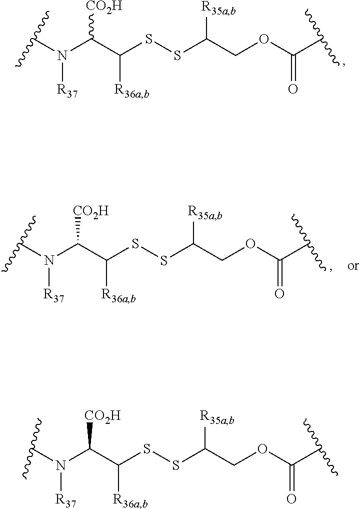

wherein R₃₇ is H or C₁-C₆ alkyl; R₃₅ₐ, R_{35b}, R₃₆ₐ, and R_{36b} each is independently H or C₁-C₆ alkyl.

The linker can comprise an amino acid. The linker can comprise an amino acid selected from the group consisting of Lys, Asn, Thr, Ser, Ile, Met, Pro, His, Gln, Arg, Gly, Asp, Glu, Ala, Val, Phe, Leu, Tyr, Cys, and Trp. The linker can comprise at least two amino acids independently selected from the group consisting of Glu and Cys. The linker can comprise Glu-Glu, wherein the glutamic acids are covalently bonded to each other through the carboxylic acid side chains.

The linker can comprise one or more hydrophilic spacer linkers comprising a plurality of hydroxyl functional groups.

One example of a linker is a linker having at least one 2,3-diaminopropionic acid group, at least one glutamic acid group (e.g., unnatural amino acid D-Glutamic acid), and at least one cysteine group. One example of such a linker is one having the non-natural amino acid, such as a linker having the repeating unit of the formula:

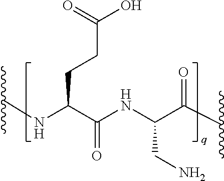

wherein q is an integer from 1 to 10 (e.g., 1 to 3 and 2 to 5), such as the linker of the general formula:

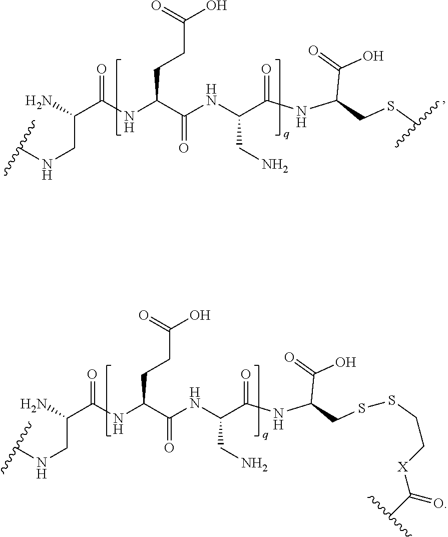

wherein X can be O, NH, NR, or S, and q is an integer from 1 to 10, or the linker of the formula:

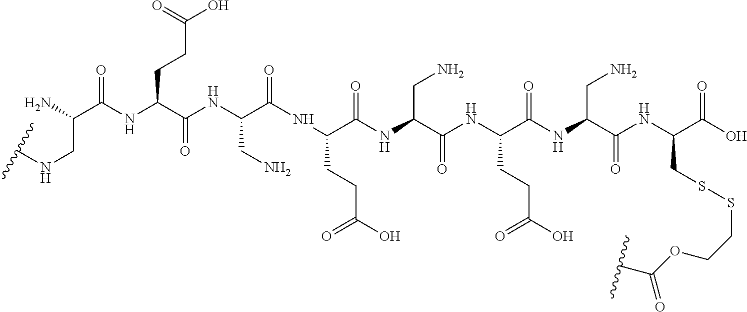 [SMILES: *NC[C@H](N)C(=O)N[C@@H](CCC(=O)O)C(=O)N[C@@H](CN)C(=O)N[C@@H](CCC(=O)O)C(=O)N[C@@H](CN)C(=O)N[C@@H](CCC(=O)O)C(=O)N[C@@H](CN)C(=O)N[C@H](CSSCCOC(*)=O)C(=O)O]

wherein the disulfide group is a part of a self-immolative group that can be generically described as a group of the formula —CH₂—S—S—CH₂.

The compounds described herein can include linkages that cause portions of the compounds described herein (e.g., NLS) to be released by any suitable mechanism, including a release mechanism involving reduction or hydrolysis. An example of a reduction mechanism includes reduction of a disulfide group into two separate sulfyhydryl groups. Thus, for example, a group of the formula —CH₂—S—S—CH₂— would be reduced to two separate groups of the formula —CH₂—SH, such that if the linker were of the formula:

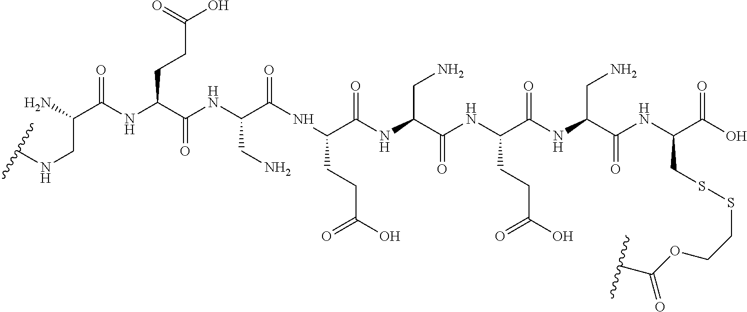 [SMILES: *NC[C@H](N)C(=O)N[C@@H](CCC(=O)O)C(=O)N[C@@H](CN)C(=O)N[C@@H](CCC(=O)O)C(=O)N[C@@H](CN)C(=O)N[C@@H](CCC(=O)O)C(=O)N[C@@H](CN)C(=O)N[C@H](CSSCCOC(*)=O)C(=O)O]

the reduction product would be of the formula:

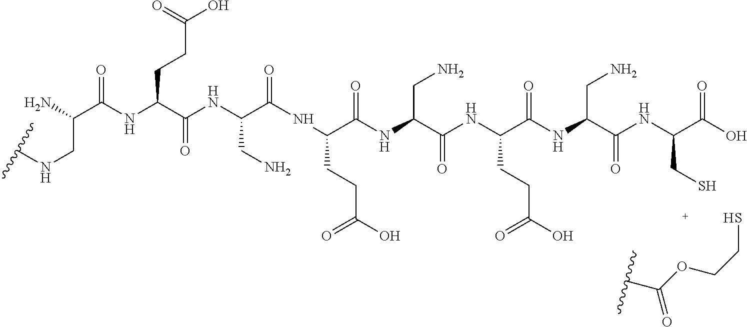 [SMILES: *NC[C@H](N)C(=O)N[C@@H](CCC(=O)O)C(=O)N[C@@H](CN)C(=O)N[C@@H](CCC(=O)O)C(=O)N[C@@H](CN)C(=O)N[C@@H](CCC(=O)O)C(=O)N[C@@H](CN)C(=O)N[C@H](CS)C(=O)O ; *C(=O)OCCS]

In this example, the NLS can be attached to the linker via a self-immolative moiety (e.g., a disulfide group).

The compounds described herein can include linkages where, for example, the NLS or X² can attached to the linker via an ester, an amide, phosphate, oxime, acetal, pyrophosphate, polyphosphate, disulfide, sulfate, hydrazide, imine, carbonate, carbamate or enzyme-cleavable amino acid sequence.

In compounds of the Formula (I), G¹ and G² represent a forward primer binding site and a reverse primer binding site, respectively. The forward primer binding site and a reverse primer binding site can be any suitable forward primer binding site and a reverse primer binding site. See, e.g., Biotechnol. Lett. 35:1541-1549 (2013). In some instances, at least one of the forward primer binding site and the reverse primer binding sites can each be pseudo-randomly generated or randomly generated. Further, in some instances, at least one of the forward primer binding, the reverse primer binding sites, and the primers themselves can be chemically modified to incorporate, among other things, functional units such as biotin, fluorescent reporters, click chemistry components, and the like. These “functional units” can be used to, among other things, differentiate between various different molecules of the Formula (I) as they are synthesized or differentiate between primers. Thus, for example, one molecule of the Formula (I) (M₁) could have at least one of the forward primer binding and the reverse primer binding sites chemically modified with a fluorescent reporter that fluoresces at one wavelength (λ₁), while a different molecule of the Formula (I) (M₂) could have at least one of the forward primer binding and the reverse primer binding sites chemically modified with a second fluorescent reporter that fluoresces at a second wavelength (λ₂) that is higher or lower than λ₁. That way, upon separation, one can easily differentiate between molecules M₁ and M₂ based on their differing fluorescence.

As used herein, the term phrase “pseudo-randomly generated” means that the sequences are randomly generated with certain design characteristics being taken into consideration. The sequence of base pairs of the forward primer binding site and/or the reverse primer binding site. There are a number of design characteristics that can be considered when generating the sequences. Examples of these considerations include, but are not limited to: the GC content of the sequence; a lack of identity to any known, naturally occurring sequences, or PCR-amplifiable region; and the lack of repetitive regions of the same base pair. The pseudo-randomly generated sequence can be designed by considering any combination of these various characteristics. As used herein, the phrase “lack of identity to any known, naturally occurring sequences” means that the sequence of base pairs of the forward primer binding site and the reverse primer binding site are designed such that they should not hybridize to naturally-occurring nucleotide sequences in a PCR-amplifiable region of the genome of a single organism. Thus the randomly generated primers should in combination (forward and reverse) not typically be capable of amplification of a naturally-occurring sequence at the hybridization temperatures and polymerase extension times typically used in real-time PCR.

In some instances, the sequences of the forward primer binding site and/or the reverse primer binding site do not have a string of more than four (4) or no more than five (5) bases that are the same in the sequence.

In the compounds described herein, Q represents RNA or a randomized single-stranded DNA. The RNA or the randomized single-stranded DNA can comprise at least 5, at least 10, at least 20, at least 40 at least 60, at least 80, at least 100, at least 120 nucleotides; or about 10 to about 100 nucleotides, about 10 to about 50 nucleotides, about 25 to about 125 nucleotides, about 10 to about 40 nucleotides, about 10 to about 20 nucleotides or about 20 to about 60 nucleotides.

In the compounds described herein, at least one of G¹, Q, and G² comprises at least one bonding arrangement that renders at least one of G¹, Q, and G² nuclease (e.g., endo- and/or exonuclease) resistant. For example, G¹ comprises at least one bonding arrangement that renders G¹ nuclease resistant; Q comprises at least one bonding arrangement that renders Q nuclease resistant; G² comprises at least one bonding arrangement that renders G² nuclease resistant; G¹ and Q comprise at least one bonding arrangement that renders G¹ and Q nuclease resistant; G¹ and G² comprise at least one bonding arrangement that renders G¹ and G² nuclease resistant; Q and G² comprise at least one bonding arrangement that renders Q and G² nuclease resistant; or G¹, Q, and G² comprise at least one bonding arrangement that renders G¹, Q, and G² nuclease resistant. Bonding arrangements that would renders at least one of G¹, Q, and G² nuclease resistant include, but are not limited to 5′-phosphorothioate, 5′-modified uracil, 4′-thio, 2′-fluoro, 5′-α-P-borano, 2′-amino, 2′-deoxy-L-ribose, 2′-methoxy, capping with 3′ end with inverted thymidine, bridged nucleic acids (BNAs), locked nucleic acids (LNAs), and xeno nucleic acids (XNAs). See, e.g., U.S. Pat. No. 9,765,328, which is incorporated by reference herein in its entirety. Examples of bonding arrangements that renders at least one of G¹, Q, and G² nuclease resistant include substitutions such as 2′-OMe, 2′-F, and 2′-methoxyethyl derivatives:

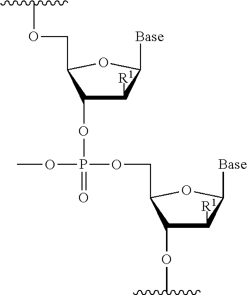

wherein each R¹ is —NH₂, —OCH₃, —(CH₂)₂OCH₃, —F (e.g., 2′-deoxy-2′-fluoro-β-D-arabino nucleic acid (2′F-ANA)) or any other group that would render at least one of G¹, Q, and G² nuclease resistant; as well as more diverse structures like locked nucleic acids (LNA), such as:

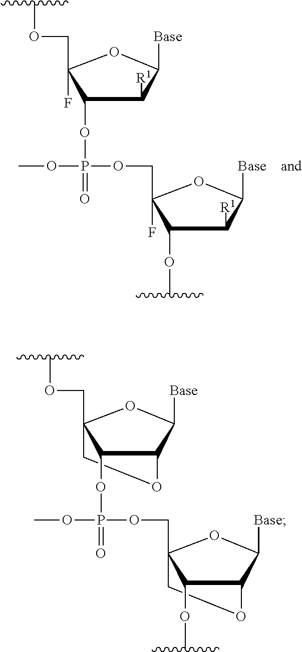

and structures such as:

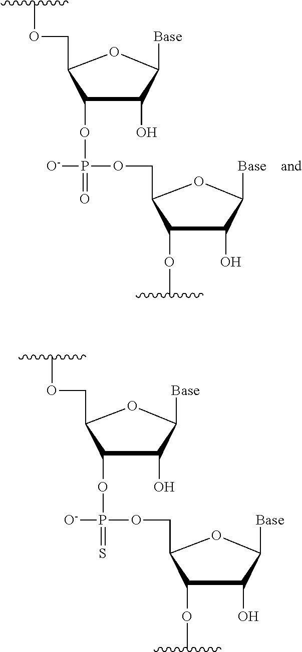

The disclosure also relates to compounds of the Formula (II), which are compounds of the Formula (I) further comprising a nuclear localization signal (NLS), such that the compound of the Formula (I) is a compound of the Formula (II):

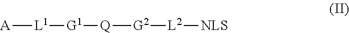

The disclosure also relates to compounds of the Formula (III), which are compounds of the Formula (I) further comprising a nuclear localization signal (NLS) and further comprising a cell penetrating peptide (CPP) such that the compound of the Formula (I) is a compound of the Formula (III):

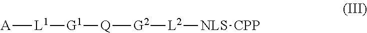

The disclosure also relates to compounds of the Formula (IV):

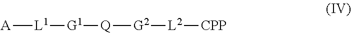

The compounds of the Formula (II)-(IV) can further comprise cell transfection molecules, enzymes, nuclear export inhibitors, and the like, in addition to a CPP or in place of a CPP.

The compounds of the Formula (II)-(IV) can further comprise at least one, at least two, at least three or four biotins, or an analog thereof, bound to at least one of the avidin-type molecule.

Also contemplated herein are compounds of the formula L²-CPP-NLS, L²-CPP, and L²-NLS, which can be, among other things, building blocks of any of the compounds described herein, such as the compounds of the Formula (II)-(IV). The L¹ and L² groups can be the same or different in any of the compounds of the Formula (II)-(IV).

Those of skill in the art will recognize that G¹ can be attached to the 5′-end of Q and G² can be attached to the 3′-end of Q or G¹ can be attached to the 3′-end of Q and G² can be attached to the 5′-end of Q.

The term “nuclear localization signal” or “nuclear localization sequence” (NLS) generally refers to an amino acid sequence that is imported into the nucleus of a cell via nuclear transport. Examples of NLSs include, but are not limited to, PKKKRKV (SEQ ID NO:1), PAAKRVKLD (SEQ ID NO:2), K(K/R)X(K/R) (SEQ ID NO:3), KRPAATKKAGQAKKKK (SEQ ID NO:4). Additional NLSs can be found in the NLS database described in Nair et al., Nucleic Acid Res., 2003, 1(31):397-9, which is incorporated by reference as if fully set forth herein. See also Published U.S. Appl. No. 2018/0346531 A1, which is incorporated by reference as if fully set forth herein.

The term “cell penetrating peptide” (CPP) generally refers to peptides of less than 30 amino acids either derived from proteins or from chimeric sequences. They are sometimes amphipathic and possess a net positive charge. CPPs can penetrate biological membranes, to trigger the movement of molecules such as the ones described herein across cell membranes into the cytoplasm and to improve their intracellular routing, thereby facilitating interactions with a target. Examples of CPPs include, but are not limited to, RRRRRRRR (SEQ ID NO:5), VEPEP-3 (e.g., Ac-X1KWFERWFREWPRKRR-cysteamide; SEQ ID NO:6), VEPEP-4 (e.g., X1WWRLSLRWW (SEQ ID NO:7), X1WFRLSLRFWR (SEQ ID NO:8), X1WWRLRSWFR (SEQ ID NO:9), and X1WFRLSLRFW (SEQ ID NO:10), wherein X1 is beta-A or S), VEPEP-6 (e.g., Ac-X1LFRALWRLLRSLWRLLWK-cysteamide; SEQ ID NO:11), VEPEP-9 (e.g., Ac-X1LRWWLRWASRWFSRWAWWR-cysteamide; SEQ ID NO:12), CADY (SEQ ID NO:13), MPG, PEP-1, PPTG 1 (SEQ ID NO:14), and poly Arginine motif. See, e.g., U.S. Pat. No. 10,189,876, which is incorporated by reference as if fully set forth herein. See also U.S. Pat. Nos. 9,376,468; 9,579,395; 9,598,465; and 9,834,581, all of which are incorporated by reference as if fully set forth herein. Although NLS can be directly attached to CPP, a linker, L³, can link NLS to CPP. The linker L³ can be any suitable linker as the term is defined herein and can be the same or different as any other linker present, such as L¹, L², and the like.

Molecules comprising CPPs are of interest herein because CPPs are not only capable to translocate themselves across membranes but also allow carrier drugs to translocate across plasma membrane, by different mechanisms depending on the CPP. Oncotarget 9:37252-37267 (2018), which is incorporated by reference as if fully set forth herein with regard to not only examples of CPPs, but also for examples of CPPs designed for preclinical and clinical cancer diagnosis and treatment including, but not limited to RI-Tat-9, TAT, MPG, BR2, and p28, with various cargoes. CPPs are also of interest here because they have been shown to have reduced cytotoxicity but also because proteins, imaging reagents, and drugs, specifically anti-cancer drugs, can be linked with these peptides and cross plasma membrane in receptor independent manner. Id. CPPs have been successfully used to transport various types of drugs, liposomes, and nanoparticles for imaging and cancer therapeutics.

While not wishing to be bound by any specific theory, it is believed that energy dependent endocytosis is the main route of entry for internalization for many CPPs. Internalization of CPP by endocytosis includes various models such as macropinocytosis, clathrin-mediated endocytosis, and caveolin-mediated endocytosis. Which of these pathways will play a role at particular time depends mostly on the basis of how big the cargo molecule is and what is its physiochemical properties. In order for CPP to reach at target site and avoid degradation from the lysosomes present in endosomes, CPP must escape from the endosome to cytosol. In one approach, pH sensitive domains can be introduced in the peptide sequence of the CPP to disrupt lipid membrane at low pH to facilitate the CPP escape from vesicles. In another approach, histidine residues can be introduced into CPPs that increase osmotic pressure in the endosomal vesicle due to proton sponge effect and eventually allow the endosomal membrane to rupture. In yet another approach, PepFect (PF) peptide alteration by N-terminal stearylation can be used to promote the endosomal escape. The presence of lysosomotropic agent chloroquine CQ equivalent was also shown to be important for improved endosomal escape. Examples of CPPs include, but are not limited to, MPG (GALFLGFLGAAGSTMGAWSQPKKKRKV (SEQ ID NO:15); amphiphilic lysine rich domain obtained from nuclear localization sequence (NLS)); Pep-1 (KETWWETWWTEWS QPKKKRKV (SEQ ID NO:16); similar to MPG and efficiently delivers wide range of peptides and proteins); Pep-2 (KETWFETWFTEWSQPKKKRKV (SEQ ID NO:17); amphipathic peptide that possesses and potency higher stability than Pep-1); Pep-3 (KETWFETWFTEWSQPKKKRKV (SEQ ID NO:18); can be used to form nanosize complexes and can have improved cellular uptake); CADY (Ac-GLWRALWRLLRSLWRLLWRA-Cya (SEQ ID NO:19); secondary amphiphillic peptide that is based on the PPTG1), and Rath (TPWWRLWTKWHHKRRDLPRKPE (SEQ ID NO:20)).

The disclosure also relates to compounds of the Formula (V):

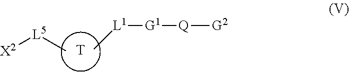

wherein L¹ and L⁵ are linker groups, as the term “linker group” is defined herein for, e.g., L¹, wherein L¹ and L⁵ can be the same or different or L⁵ can be a bond; T is a click-chemistry-derived core; G¹ is a forward primer binding site; Q is a randomized single-stranded DNA; and G² is a reverse primer binding site. In some instances, L⁵ is a bond. Examples of click-chemistry-derived cores include triazoles, oxazoles, and other heterocycles that can be derived from, e.g., electrocyclizations including Diels-Alder electrocyclizations. Some examples of click-chemistry-derived cores include those shown in Schemes 7-12:

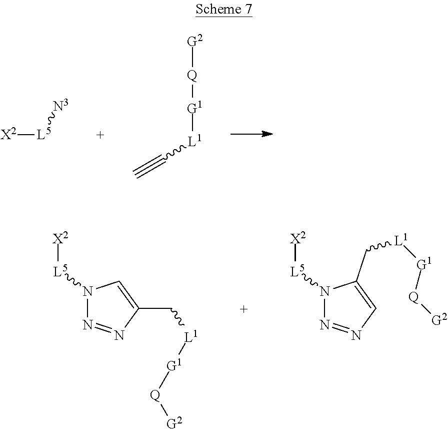

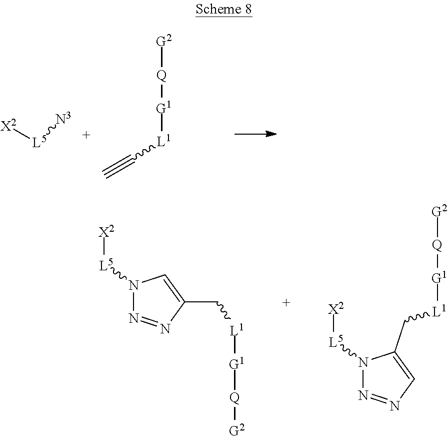

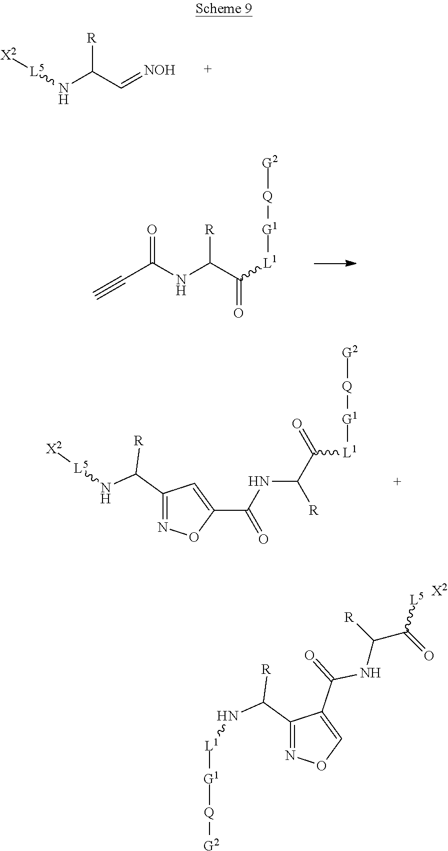

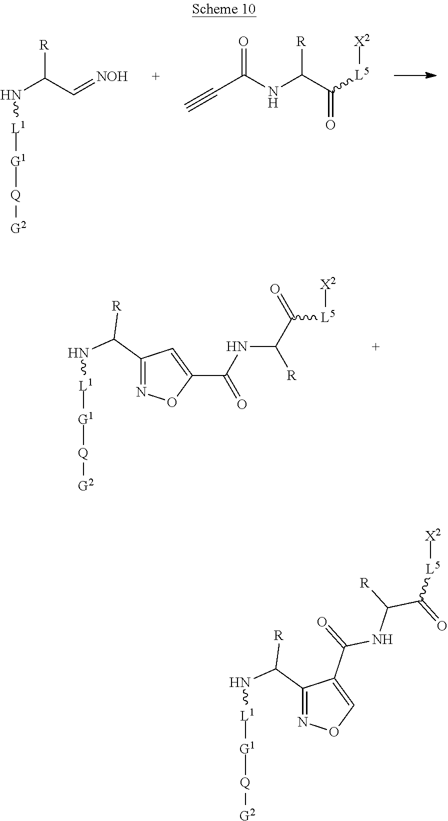

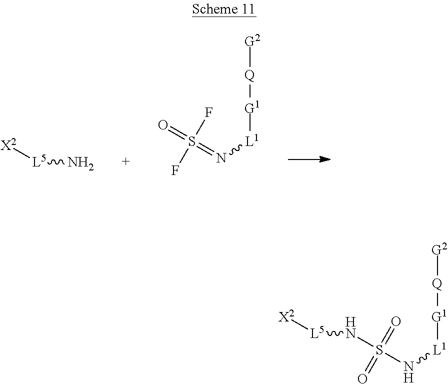

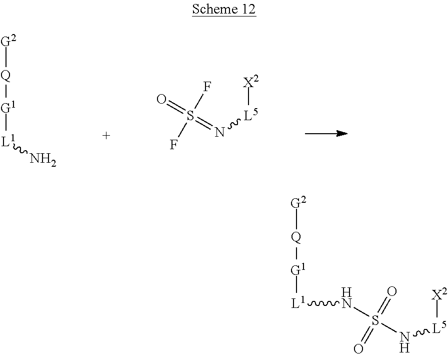

Such compounds can also be accessed via so-called inverse-electron-demand Diels-Alder chemistry from a tretrazine and trans-cyclooctene as shown in Scheme 13:

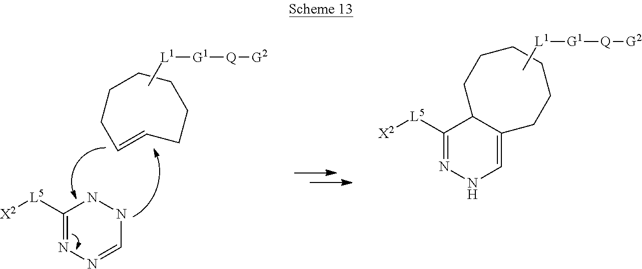

Examples of compounds that can be accessed via inverse-electron-demand Diels-Alder chemistry from a tretrazine and trans-cyclooctene include compounds of the Formulae:

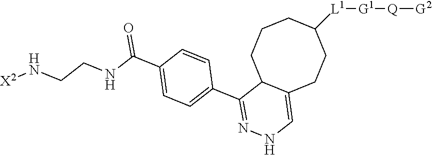

such as

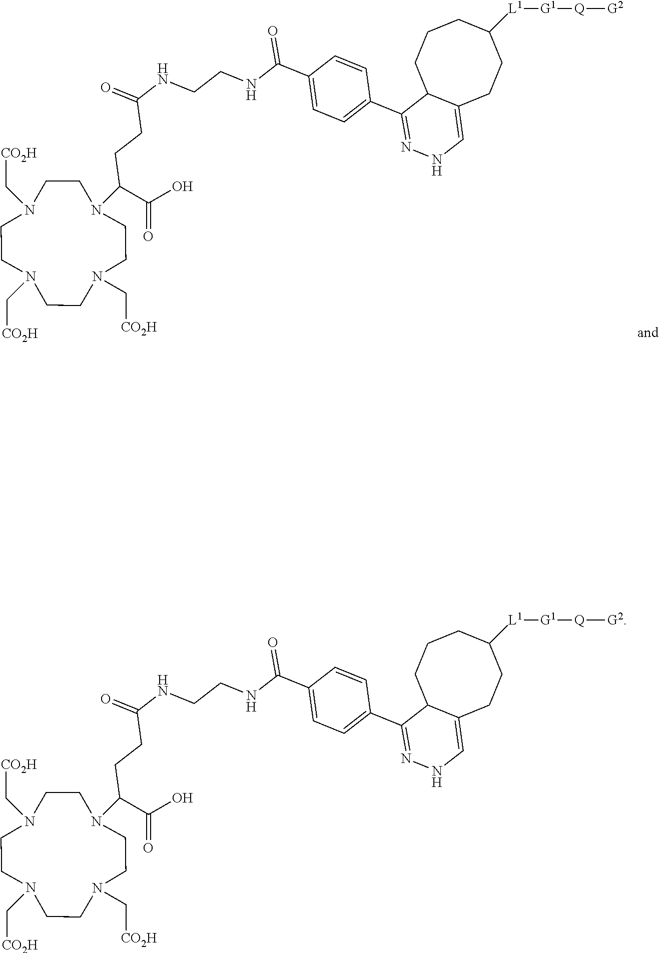

The disclosure also relates to compounds of the Formula (VI), which are compounds of the Formula (V) further comprising a nuclear localization signal (NLS), such that the compound of the Formula (V) is a compound of the Formula (VI):

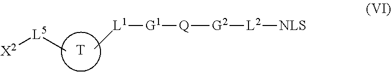

The disclosure also relates to compounds of the Formula (VI), which are compounds of the Formula (V) further comprising a nuclear localization signal (NLS) and further comprising a cell penetrating peptide (CPP) such that the compound of the Formula (V) is a compound of the Formula (VII):

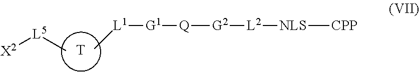

The disclosure also relates to compounds of the Formula (VIII):

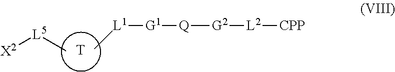

In any of the compounds described herein, such as the compounds of Formulae (V)-(VIII) and the compounds described in Schemes 7-13, it is contemplated that X² can be an antibody or a fragment thereof, such as an Fc fragment. Such compounds are envisioned to be useful in a form of immune therapy by recruiting an immune effector response. Thus, for example, it is envisioned that X² can have one or more tetrazine molecules attached to it (e.g., covalently) such that one or more selected oligonucleotides can either be directly attached to X² or be attached to X² via a suitable linkage (e.g., L¹ or L⁵) to the oligonucleotides described herein (e.g., at least one of G¹, G², and Q). Therefore, the resulting compound shown in, e.g., Scheme 13, be used to target cancer cells or pathogens (e.g., virion, bacteria, and prions).

The disclosure also provides a pharmaceutical composition comprising a compound of any of the preceding formulae and a pharmaceutically acceptable carrier. The disclosure also provides a pharmaceutical composition comprising a therapeutically effective amount of a compound of one of Formulae (I)-(VIII), and a pharmaceutically acceptable carrier.

A “pharmaceutical composition” refers to a chemical or biological composition suitable for administration to a subject (e.g., mammal). Such compositions can be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” is a carrier, sometimes a liquid, in which an active therapeutic agent is formulated. The excipient generally does not provide any pharmacological activity to the formulation, though it can provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes, but is not limited to, any and all solvents, dispersion media, coatings, antibacterial and antifungal agents, isotonic and absorption delaying agents that are physiologically compatible. The carrier can be suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyglycolic polyanhydrides, acid, collagen, polyorthoesters, polylactic acid and polylactic, polyglycolic copolymers (PLG). Many methods for the preparation of such formulations are known to those skilled in the art.

Oral forms of administration are also contemplated herein. Pharmaceutical compositions can be orally administered as a capsule (hard or soft), tablet (film coated, enteric coated or uncoated), powder or granules (coated or uncoated) or liquid (solution or suspension). Formulations can be conveniently prepared by any of the methods well-known in the art. Pharmaceutical compositions can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

The compounds described herein can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition can vary according to factors such as the disease state, age, gender, weight, patient history, risk factors, predisposition to disease, administration route, pre-existing treatment regime (e.g., possible interactions with other medications), and weight of the subject. Dosage regimens can be adjusted to provide the optimum therapeutic response. For example, a single bolus can be administered, several divided doses can be administered over time, or the dose can be proportionally reduced or increased as indicated by the exigencies of therapeutic situation.

A “dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on the unique characteristics of the active compound and the particular therapeutic effect to be achieved, and the limitations inherent in the art of compounding such an active compound for the treatment of sensitivity in subjects. In therapeutic use for treatment of conditions in mammals (e.g., humans) for which the compounds described herein or an appropriate pharmaceutical composition thereof are effective, the compounds can be administered in an effective amount. The dosages as suitable can be a composition, a pharmaceutical composition or any other compositions described herein.

The dosage is typically administered once, twice, or thrice a day, although more frequent dosing intervals are possible. The dosage can be administered every day, every 2 days, every 3 days, every 4 days, every 5 days, every 6 days, and/or every 7 days (once a week). The dosage can be administered daily for up to and including 30 days, preferably between 7-10 days. The dosage can be administered twice a day for 10 days. If the patient requires treatment for a chronic disease or condition, the dosage can be administered for as long as signs and/or symptoms persist. The patient can require “maintenance treatment” where the patient is receiving dosages every day for months, years, or the remainder of their lives. In addition, the composition can be to effect prophylaxis of recurring symptoms. For example, the dosage can be administered once or twice a day to prevent the onset of symptoms in patients at risk, especially for asymptomatic patients.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to, for example, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

The term “therapeutically effective amount” as used herein, refers to that amount of one or more compounds described herein that elicits a biological or medicinal response in a tissue system, animal or human, that is being sought by a researcher, veterinarian, medical doctor or other clinician, which includes alleviation of the symptoms of the disease or disorder being treated. In some examples, the therapeutically effective amount is that which can treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

The term “alkyl” as used herein refers to substituted or unsubstituted straight chain, branched and cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopently, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Alkyl can include a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. Alkyl can be methyl and C₂-C₈ linear alkyl. Alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Representative substituted alkyl groups can be substituted from a set of groups including amino, hydroxy, cyano, carboxy, nitro, thio and alkoxy, but not including halogen groups. Thus, for example, alkyl can be substituted with a non-halogen group. For example, representative substituted alkyl groups can be substituted with a fluoro group, substituted with a bromo group, substituted with a halogen other than bromo, or substituted with a halogen other than fluoro. Representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl.

The term “alkenyl” as used herein refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)alkenyl groups include those with from 2 to 6 carbon atoms such as —CHCH—, —CHCHCH₂—, —CHCHCH₂CH₂—, and—CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. Alkenyl can be understood to include a combination of substituted and unsubstituted alkenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Representative substituted alkenyl groups can be substituted from a set of groups including monoalkylamino, dialkylamino, cyano, acetyl, amido, carboxy, nitro, alkylthio and alkoxy, but not including halogen groups. Thus, for example, alkenyl can be substituted with a non-halogen group. Representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. Representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. Heterocyclyl groups include 3 to about 20 ring members, whereas other such groups have 3 to about 15 ring members. Heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring can be an example of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

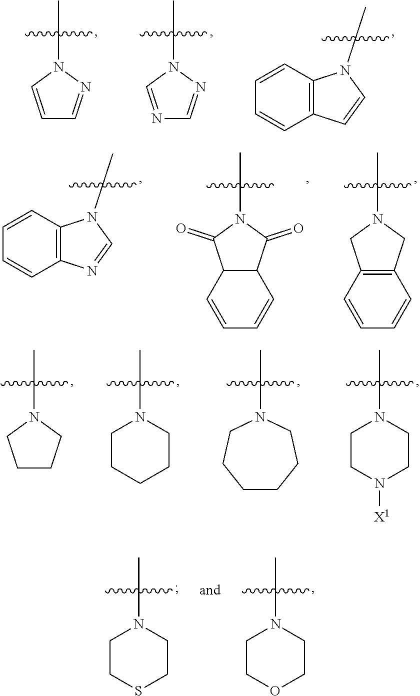

wherein X¹ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl) alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” or “halide” group, as used herein, by themselves or as part of another substituent, mean, unless otherwise stated, a fluorine, chlorine, bromine, or iodine atom.

The term “amino” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, alkoxy, aryl, cycloalkyl, heterocyclyl, group or the like.

The term “formyl” as used herein refers to a group containing a carbonyl moiety wherein the group is bonded via the carbonyl carbon atom. The carbonyl carbon atom is also bonded to a hydrogen atom.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “arylcarbonyl” as used herein refers to a group containing a carbonyl moiety wherein the group is bonded via the carbonyl carbon atom. The carbonyl carbon atom is also bonded to an aryl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to an nitrogen atom which is bonded to one or more aryl group instead of, or in addition to, the one or more alkyl group. In a further case, which is also an alkylamido as the term is defined herein, the carbonyl carbon atom is bonded to an nitrogen atom which is bonded to one or more alkenyl group instead of, or in addition to, the one or more alkyl and or/aryl group. In a further case, which is also an alkylamido as the term is defined herein, the carbonyl carbon atom is bonded to an nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “alkylthio” as used herein refers to a sulfur atom connected to an alkyl, alkenyl, or alkynyl group as defined herein.

The term “arylthio” as used herein refers to a sulfur atom connected to an aryl group as defined herein.

The term “alkylsulfonyl” as used herein refers to a sulfonyl group connected to an alkyl, alkenyl, or alkynyl group as defined herein.

The term “alkylsulfinyl” as used herein refers to a sulfinyl group connected to an alkyl, alkenyl, or alkynyl group as defined herein.

The term “dialkylaminosulfonyl” as used herein refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups in place of the alkyl groups.

The term “dialkylamino” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups.

The term “dialkylamido” as used herein refers to an amido group connected to two alkyl groups, as defined herein, and which can optionally be linked together to form a ring with the nitrogen. This term also includes the group where the nitrogen is further connected to one or two alkenyl groups in place of the alkyl groups.

The term “substituted” as used herein refers to a group that is substituted with one or more groups (substituents) including, but not limited to, the following groups: deuterium (D), halogen (e.g., F, Cl, Br, and I), R, OR, OC(O)N(R)₂, CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl or (C₆-C₂₀)aryl. Substituted also includes a group that is substituted with one or more groups including, but not limited to, the following groups: fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

The compounds described herein (e.g., the compounds of the Formulae (I)-(VI)) can contain chiral centers. All diastereomers of the compounds described herein are contemplated herein, as well as racemates.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric;

and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a described herein. Examples of prodrugs include, but are not limited to, derivatives and metabolites of a compound described herein that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

As used herein, the term “subject” or “patient” refers to any organism to which a composition described herein can be administered, e.g., for experimental, diagnostic, prophylactic and/or therapeutic purposes. Subject refers to a mammal receiving the compositions disclosed herein or subject to disclosed methods. It is understood and herein contemplated that “mammal” includes but is not limited to humans, non-human primates, cows, horses, dogs, cats, mice, rats, rabbits, and guinea pigs.

The disclosure also relates to methods of treatment in a subject in need of such treatment, the method comprising administering a therapeutically-effective amount of one or more compounds of Formulae (I)-(VI). Thus, for example, the disclosure encompasses a method of treating cancer in a subject in need of such treatment, the method comprising administering a therapeutically-effective amount of one or more compounds of Formulae (I)-(VI). The types of cancer that can be alleviated or treated include, but are not limited to prostate cancer, breast cancer, pancreatic cancer, thyroid cancer, bone cancer, glioblastoma and neuroendocrine tumors. The compounds of the Formulae (I)-(VIII) can be administered in combination with at least one anticancer agent. Anticancer agents include those described herein, as well as Fluoropyrimidines-5-FU, Fluorodeoxyuridine, Ftorafur, 5′-deoxyfluorouridine, UFT, S-1 Capecitabine; pyrimidine Nucleosides-Deoxycytidine, Cytosine Arabinoside, 5-Azacytosine, Gemcitabine, 5-Azacytosine-Arabinoside; Purines-6-Mercaptopurine, Thioguanine, Azathioprine, Allopurinol, Cladribine, Fludarabine, Pentostatin, 2-Chloro Adenosine; Platinum Analogues-Cisplatin, Carboplatin, Oxaliplatin, Tetraplatin, Platinum-DACH, Ormaplatin, CI-973, JM-216; Anthracyclines/Anthracenediones-Doxorubicin, Daunorubicin, Epirubicin, Idarubicin, Mitoxantrone; Epipodophyllotoxins—Etoposide, Teniposide; Camptothecins—Irinotecan, Topotecan, Lurtotecan, Silatecan, 9-Amino Camptothecin, 10, 11-Methylenedioxy Camptothecin, 9-Nitro Camptothecin, TAS 103, 7-(4-methyl-piperazino-methylene)-10,11-ethylenedioxy-20(S)-camptothecin, 7-(2-N-isopropylamino)ethyl)-20(S)-camptothecin; Hormones and Hormonal Analogues-Diethylstilbestrol, Tamoxifen, Toremefine, Tolmudex, Thymitaq, Flutamide, Bicalutamide, Finasteride, Estradiol, Trioxifene, Droloxifene, Medroxyprogesterone Acetate, Megesterol Acetate, Aminoglutethimide, Testolactone and others; Enzymes, Proteins and Antibodies—Asparaginase, Interleukins, Interferons, Leuprolide, Pegaspargase, and others; Vinca Alkaloids—Vincristine, Vinblastine, Vinorelbine, Vindesine; Taxanes—Paclitaxel, Docetaxel; Anastrozole; Antifolates—Methotrexate, Aminopterin, Trimetrexate, Trimethoprim, Pyritrexim, Pyrimethamine, Edatrexate, MDAM; Antimicrotubule Agents—Taxanes and Vinca Alkaloids; Alkylating Agents (Classical and Non-Classical)—Nitrogen Mustards (Mechlorethamine, Chlorambucil, Melphalan, Uracil Mustard), Oxazaphosphorines (Ifosfamide, Cyclophosphamide, Perfosfamide, Trophosphamide), Alkylsulfonates (Busulfan), Nitrosoureas (Carmustine, Lomustine, Streptozocin), Thiotepa, Dacarbazine and others; Antimetabolites—Purines, pyrimidines and nucleosides, listed above; Antibiotics—Anthracyclines/Anthracenediones, Bleomycin, Dactinomycin, Mitomycin, Plicamycin, Pentostatin, Streptozocin; topoisomerase Inhibitors—Camptothecins (Topo I), Epipodophyllotoxins, m-AMSA, Ellipticines (Topo II); Antivirals-AZT, Zalcitabine, Gemcitabine, Didanosine, and others; Miscellaneous Cytotoxic Agents—Hydroxyurea, Mitotane, Fusion Toxins, PZA, Bryostatin, Retinoids, Butyric Acid and derivatives, Pentosan, Fumagillin, and others. The small molecule may be an anthracycline drug, doxorubicin, daunorubicin, mitomycin C, epirubicin, pirarubicin, rubidomycin, carcinomycin, N-acetyladriamycin, rubidazone, 5-imidodaunomycin, N-acetyldaunomycine, daunoryline, mitoxanthrone; a camptothecin compound, camptothecin, 9-aminocamptothecin, 7-ethylcamptothecin, 10-hydroxycamptothecin, 9-nitrocamptothecin, 10,11-methyl enedioxyc amptothecin, 9-amino-10,11-methylenedioxycamptothecin, 9-chloro-10,11-methylenedioxycamptothecin, irinotecan, topotecan, lurtotecan, silatecan, (7-(4-methylpiperazinomethylene)-10,11-ethylenedioxy-20(S)-camptothecin, 7-(4-methylpiperazinomethylene)-10,11-methylenedioxy-20(S)-camptothecin, 7-(2-N-isopropylamino)ethyl)-(20S)-camptothecin; an ellipticine compound, ellipticine, 6-3-aminopropyl-ellipticine, 2-diethylaminoethyl-ellipticinium and salts thereof, datelliptium, retelliptine; topoisomerase inhibitor, vinca alkaloid, e.g., vincristine, vinblastine, vinorelbine, vinflunine, and vinpocetine, microtubule depolymerizing or destabilizing agent, microtubule stabilizing agent, e.g., taxane, aminoalkyl or aminoacyl analog of paclitaxel or docetaxel, e.g., 2′-[3-(N,N-diethylamino)propionyl]paclitaxel, 7-(N,N-dimethylglycyl)paclitaxel, and 7-L-alanylpaclitaxel, alkylating agent, receptor-binding agent, tyrosine kinase inhibitor, phosphatase inhibitor, cycline dependent kinase inhibitor, enzyme inhibitor, aurora kinase inhibitor, nucleotide, polynicleotide, and famesyltransferase inhibitor.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

Each embodiment described above is envisaged to be applicable in each combination with other embodiments described herein. For example, embodiments corresponding to Formula (I) are equally envisaged as being applicable to Formulae (II) and (III). As another example, embodiments corresponding to Formula (II) are equally envisaged as being applicable to Formulae (I) and (III) and so on and so forth.

The term “about” as used herein can allow for a degree of variability in a value or range, for example, within 10%, within 5%, or within 1% of a stated value or of a stated limit of a range.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### EXAMPLES

The present invention can be better understood by reference to the following examples which are offered by way of illustration. The present invention is not limited to the examples given herein.

### Example 1

The compound of the formula (IX):

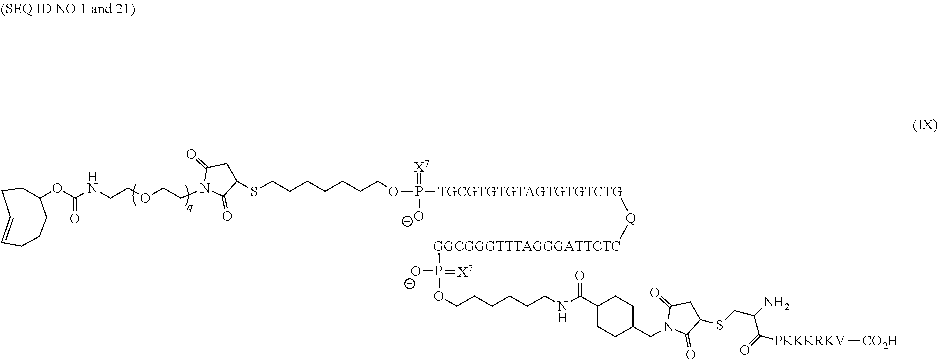

wherein X⁷ is O or S; and q is an integer that encompasses a polyethylene (PEG) group having an average Mn of 5,000; was synthesized in a stepwise fashion. wherein q is an integer that encompasses a polyethylene (PEG) group having an average Mn of 5,000; was synthesized in a stepwise fashion. It should be understood the compounds of the formula (IX) and formula (X) herein can be nuclease (e.g., endo- and exonuclease) resistant by virtue of the oligonucleotide sequences, including Q, comprising one or more phosphorothioate bonds between nucleotides:

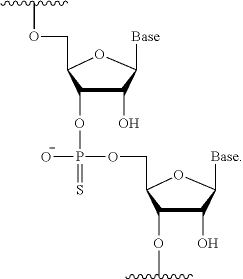

Accordingly, in one example, one or more or all of the nucleotides in the oligonucleotide sequences shown in the compounds of the formula (IX) and (X) can comprise phosphorothioate linkages. Thus, for example, the oligonucleotide sequences can comprise a sufficient number of phosphorothioate linkages to make the compounds of the formula (IX) and (X) nuclease resistant.

In one step, the NLS peptide with a cysteine residue was synthesized having the formula SHCH₂CH(NH₂)—C(O)PKKKRKV—CO₂H (SEQ ID NO:1), which will be referred to herein as “Cys-NLS.” In another step, the randomized single-stranded DNA corresponding to Q herein, was synthesized. In this example, the randomized single-stranded DNA can have the sequence TCGGGATCAACCCGAGTTCACGCAACT (SEQ ID NO:22). Other sequences Q that have been incorporated into the compounds of the formula (IX), and, in turn, into the compounds of the formula (X), include the sequences:

(SEQ ID NO: 23)
ACACTAGTACAGTCAGTACGCACGA;
(SEQ ID NO: 24)
TTTTGCGCCTGAAGCCTCCCCAGGA;
(SEQ ID NO: 25)
GTCCGTATCTTGGTCGAAGATGTAC;
(SEQ ID NO: 26)
AGGTTCAATCTACCTTCTGCCATGC;
(SEQ ID NO: 27)
CTGGAATTCCAAATATGCCGGCGAG;
(SEQ ID NO: 28)
ATCGTTCTGGGATACAAGCTTTTGA;
(SEQ ID NO: 29)
AAATATACATTATTCCCATCAAAAT;
(SEQ ID NO: 30)
GGACAAAAAGTCTGAGTCTGACCTT;
(SEQ ID NO: 31)
GATGGCAGTATAGTCGTCATGAGTC;
(SEQ ID NO: 32)
TCGGGATCAACCCGAGTTCACGCAA;
(SEQ ID NO: 33)
ACTCTTTCTGTCACAAGATCTGCAT;
(SEQ ID NO: 34)
AATGTGGATGGCATAGTGGCGGCGC;
(SEQ ID NO: 35)
TACAACCTAGAGGATGAGCTCACGA;
(SEQ ID NO: 36)
TTAGTGTTACATCACATCTCGAGCT;
(SEQ ID NO: 37)
TAGGAGGCATTCATTTTTAAGGTAG;
(SEQ ID NO: 38)
GTTACCACATCGATCTGCGAAAACT;
(SEQ ID NO: 39)
CACCAGTGGTTTGATATACGGCCTA;
(SEQ ID NO: 40)
TAGGTTGTGGTTCCCGAATCGTGAG;
(SEQ ID NO: 41)
GCAACTTGGCTTCCGTCTAAACAAA;
(SEQ ID NO: 42)
GCTATCCTTCTTTTAGCAGACAGTA;
(SEQ ID NO: 43)
TACTCGTCGCGTTTTATTTTTTTGC;
(SEQ ID NO: 44)
ATAAGCCCCCAGCTACTCCCGTTTT;
(SEQ ID NO: 45)
CTTCATGTAGCAACTCATTGTGAAG;
(SEQ ID NO: 46)
TCCCAAAATCTCGCCCCCCGGAATA;
(SEQ ID NO: 47)
AGTTGCCACAAAAATTTACTAAGTC;
(SEQ ID NO: 48)
CAGCTACATTCATTATTTGTTTCCG;
(SEQ ID NO: 49)
TCATACGGAACCTCAGCCCATGACA;
(SEQ ID NO: 50)
CACTTATTAGAAATGCATACCTATA;
(SEQ ID NO: 51)
GTCTGAAATTAATTGATCGTCATGC;
(SEQ ID NO: 52)
GCATCCGATTCACACACTCGCTCAC;
(SEQ ID NO: 53)
CATAACAGATGTTAAATTAGCGTAA;
(SEQ ID NO: 54)
AAAATCATGAGTGGTTTACCGGTAG;
(SEQ ID NO: 55)
CAACCCGCAAAAAGTCTCAGGAGTA;
(SEQ ID NO: 56)
CAGCTTCGCCATCCCTACGGGTAAG;
(SEQ ID NO: 57)
TTCTGCATGGCGGGTATACTCACTA;
(SEQ ID NO: 58)
CCCGTAGCCAAGGAGCCTATACAAC;
(SEQ ID NO: 59)
TTTAAGTTTCCGAATCCAACGTAAA;
(SEQ ID NO: 60)
GGGGAGCAGCAGCGATTTGACCTAG;
(SEQ ID NO: 61)
GAACCAAAGCGCTGCTTCCCATAAA;
(SEQ ID NO: 62)
ATGGTTATCTTACCCTACCAAAGGA;
(SEQ ID NO: 63)
GACCGGGTTCATTTAACCGTACGGG;
(SEQ ID NO: 64)
CTCCAACGCAGTAGCCAGGTACACA;
(SEQ ID NO: 65)
TAACTGTTGCCTCTCACATGGTCAA;
(SEQ ID NO: 66)
CGCTTAAGTGGTATAGTCTCCATCG;
(SEQ ID NO: 67)
TCGCACCCTATCATAGTCCGACACC;
(SEQ ID NO: 68)
GAATCTAAACGTTAGCAATCGGCGT;
(SEQ ID NO: 69)
CGGTTCCAGGTGGGCCCGTATACGT;
(SEQ ID NO: 70)
AGCTCCCCCCCGCGTTATACCTGAC;
(SEQ ID NO: 71)
GCCTAGCATCCAATCGACGTACCGC;
(SEQ ID NO: 72)
CACAACTTCCATATGTCAGTTCAAC;
(SEQ ID NO: 73)
AAGCAGTCTCATTCATCCATCACTA;
(SEQ ID NO: 74)
GCTTGGATTTAAACCAAGCGTCCCG;
(SEQ ID NO: 75)
TAGATATCGTATAATATGGGGATAA;
(SEQ ID NO: 76)
TAATGCCACACGTTATGTCTCCCAA;
(SEQ ID NO: 77)
ACGAAGGCGGCATGGTAATCTGCAC;
(SEQ ID NO: 78)
GGCGTGGGTTGACCGGATACATGAA;
(SEQ ID NO: 79)
TACAAGACCGAACCTGGTTTATACC;
(SEQ ID NO: 80)
GTGGACTCGACATCCGACAGTCAGT;
(SEQ ID NO: 81)
TAACCGGTTGGATAGCGATTCGATT;
(SEQ ID NO: 82)
GCCAGTCTAACAGTAATTATGCAAA;
(SEQ ID NO: 83)
TATTCATAATACATGAGATGGCACG;
(SEQ ID NO: 84)
CATAGGATTCGTAATGTATAAGTGT;
(SEQ ID NO: 85)
TATCTCAGAATACCGCACTCACGTG;
(SEQ ID NO: 86)
CGGTCAAAGCACCTGGAGCGTATTC;
(SEQ ID NO: 87)
CTGTACTTGATCCAAGGTTTGAAGG;
(SEQ ID NO: 88)
GACTACGTCGCTTGCAAATCATCCG;
(SEQ ID NO: 89)
TGTAGTCTATAACTCCCTGGCGCAG;
(SEQ ID NO: 90)
GGTAGGCAGCACGTTTGTGTGAACC;
(SEQ ID NO: 91)
CTCTTCACCATTTTATCGCCATGCA;
(SEQ ID NO: 92)
GTCTGTCTGTATCATCCGAGCGACA;
(SEQ ID NO: 93)
GGAAGCGGGATATATGGTGCCGTCC;
(SEQ ID NO: 94)
TAACTCAGACAGCTAGCTATCGTTA;
(SEQ ID NO: 95)
TAACTCTGCATTGCTCTCAGGGAGC;
(SEQ ID NO: 96)
GTGTCTTGTACTCTGACCTGAAGCG;
(SEQ ID NO: 97)
GGAATAAGAAGTCTTAGTAGCCCAC;
(SEQ ID NO: 98)
AAATAGTAAATTGAGGAGCCGTTTA;
(SEQ ID NO: 99)
CTACGTATAAACGGTTGGTTAGGTT;
(SEQ ID NO: 100)
AGCATGAATGGAGGCCGTTAACAAA;
(SEQ ID NO: 101)
TCCCGCAGTTATCGCGGCTGTCTCA;
(SEQ ID NO: 102)
CGCCGTAGGGCGTATGGCGCGTCTG;
(SEQ ID NO: 103)
CTACGTGCCAGTTTATACCCCGGAA;
(SEQ ID NO: 104)
CCGCTAGAGAACCTTGATGATTCTG;
(SEQ ID NO: 105)
TATCTTAAGTCAGTGGGGCTCGTCG;
(SEQ ID NO: 106)
TTTATGAAGAGCACATCATAAGAAG;
(SEQ ID NO: 107)
TGGCCGCCTAGAGTTAAGAACTATT;
(SEQ ID NO: 108)
GGGTCGAATCTAGTTTTGTAACAGG;
(SEQ ID NO: 109)
TGACAGTGGCGTCACCCGTTCACCC;
(SEQ ID NO: 110)
CTGCGTACTGGATATGTAAAAGATG;
(SEQ ID NO: 111)
GGAATGCGTGCAGACCCGTTGGTTT;
(SEQ ID NO: 112)
GTACCCAAATGTGAGTGACGCCATT;
(SEQ ID NO: 113)
CATCTCTGTGTACGGAAATCTTTGA;
(SEQ ID NO: 114)
GTTTAGCGATCCTTTTGAGCATTAG;
(SEQ ID NO: 115)
CTAAGTTGTAAGCGCAGCACAGCGT;
(SEQ ID NO: 116)
GAAACCTTTGTGCAACGCTCGTGTT;
(SEQ ID NO: 117)
CTTGTCTGTCCCGCTAGTTTGGGGG;
(SEQ ID NO: 118)
CTAAAACCGGGCGTAGACGATGGTC;
(SEQ ID NO: 119)
CGGTAGTCGCCGGCTTATATGCCGA;
(SEQ ID NO: 120)
TTCTGATATCTGATGTTTTATGGCT;
(SEQ ID NO: 121)
AGCTCGTCTAGAAACCGTGGGCCAA;
and
(SEQ ID NO: 122)
GATAAGGGTAGAGTCCAGTGAACGG,

such that complete aptamers that can be located between the two phosphates in the compounds of formula (IX) and (X) can be:

(SEQ ID NO: 123)
TGCGTGTGTAGTGTGTCTGTCGGGATCAACCCGAGTTCACGCAACTCT
TAGGGATTTGGGCGG;
(SEQ ID NO: 124)
TGCGTGTGTAGTGTGTCTGGACCGGGTTCATTTAACCGTACGGGCTCT
TAGGGATTTGGGCGG;
(SEQ ID NO: 125)
TGCGTGTGTAGTGTGTCTGGCTTGGATTTAAACCAAGCGTCCCGCTCT
TAGGGATTTGGGCGG;
and
(SEQ ID NO: 126)
TGCGTGTGTAGTGTGTCTGCGGTAGTCGCCGGCTTATATGCCGACTCT
TAGGGATTTGGGCGG.

The randomized single-stranded DNA corresponding to Q herein was synthesized to have a protected thiol group at the 5′-end and an amino group at the 3′-end. This compound will be referred to herein as “5′-proc-S-oligo-NH₂-3′.” In another step, 5′-proc-S-oligo-NH₂-3′ was functionalized with SMCC to make 5′-proc-S-oligo-Mal-3′, which was, in turn conjugated with an NLS peptide to make 5′-proc-S-oligo-NLS conjugate 5′-proc-S-oligo-linker1-NLS. The 5′-proc-S-oligo-NLS conjugate 5′-proc-S-oligo-linker1-NLS was subsequently deprotected to give a 5′-HS-oligo-NLS conjugate. In a subsequent step, 5′-HS-oligo-NLS conjugate was reacted with a compound referred to herein as “TCO-PEG-Mal,” which comprises the trans-cyclooctene (TCO) linked via a carbamyl group to the PEG group, which, in turn, is linked to the maleimide nitrogen as shown herein in the compound of formula (IX). This synthesis gave approximately 414 μg of the compound of formula (IX), which is also referred to herein as “6867SP-1.” The compound of formula (IX) had an approximate molecular weight of 27,600 Daltons. The compound of formula (IX) was shown to be stable at 95° C. for 30 minutes.

### Example 2

Although the inverse electron demand Diels-Alder reaction described herein between a tetrazine compound and a cyclooctene compound can occur rapidly under mild conditions, in the absence of a catalyst, the compound 6867SP-1 was subsequently reacted under coppercatalyzed alkyne-azide cycloaddition click reaction with the compound:

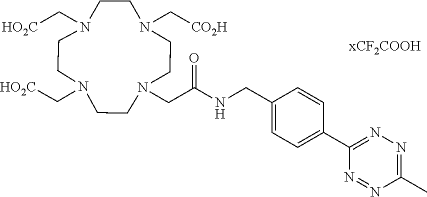 [SMILES: Cc1nnc(-c2ccc(CNC(=O)CN3CCN(CC(=O)O)CCN(CC(=O)O)CCN(CC(=O)O)CC3)cc2)nn1]

to give the compound of formula (X):

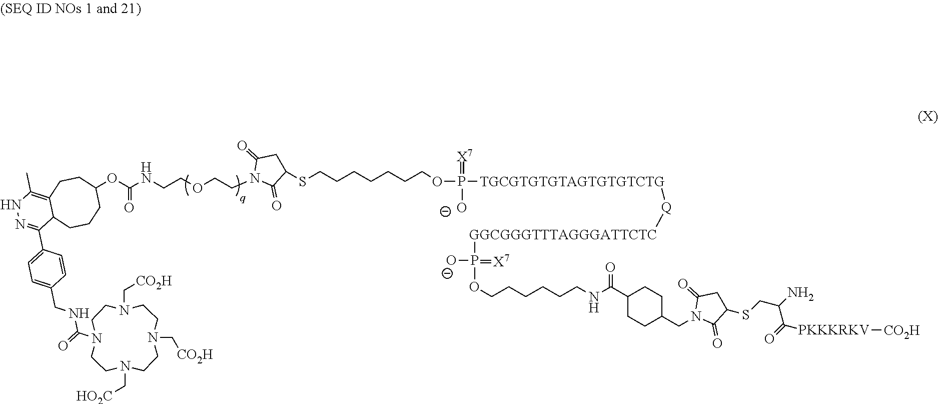

which is referred to herein as 6867SP-1 DOTA, an approximate molecular weight of 28,200 Daltons. The compound of formula (X) was shown to be stable at 95° C. for 30 minutes. Also contemplated herein are regioisomers of the formula (X) resulting from the cycloaddition click reaction between the tetrazine and the cyclooctene.

### Example 3

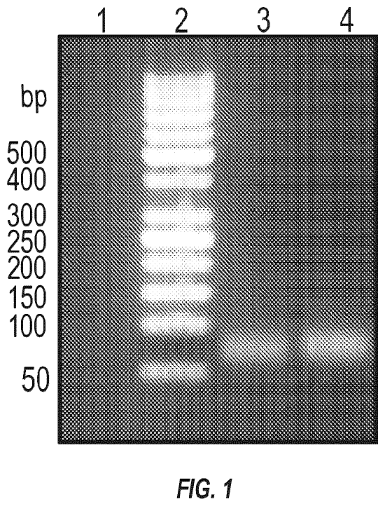

The compounds of formula (IX) and formula (X) were subjected to PCR. The PCR products were analyzed via DNA polyacrylamide gel electrophoresis using a suitable buffer (e.g., 5 x Tris-Borate-EDTA). FIG. 1 is a photograph of a gel where lane 1 corresponds to a water control; lane 2 corresponds to a doubled stranded DNA (dsDNA) standards ladder; lane 3 corresponds to the PCR product of the compound of formula (IX); and lane 4 corresponds to the PCR product of the compound of formula (X). These results demonstrate that even though the compounds of the formula (IX) and (X) have moieties conjugated at the 5′-end and at the 3′-end, the aptamer that includes Q can still undergo multiple cycles of PCR (e.g., approximately 21 cycles).

### Example 4

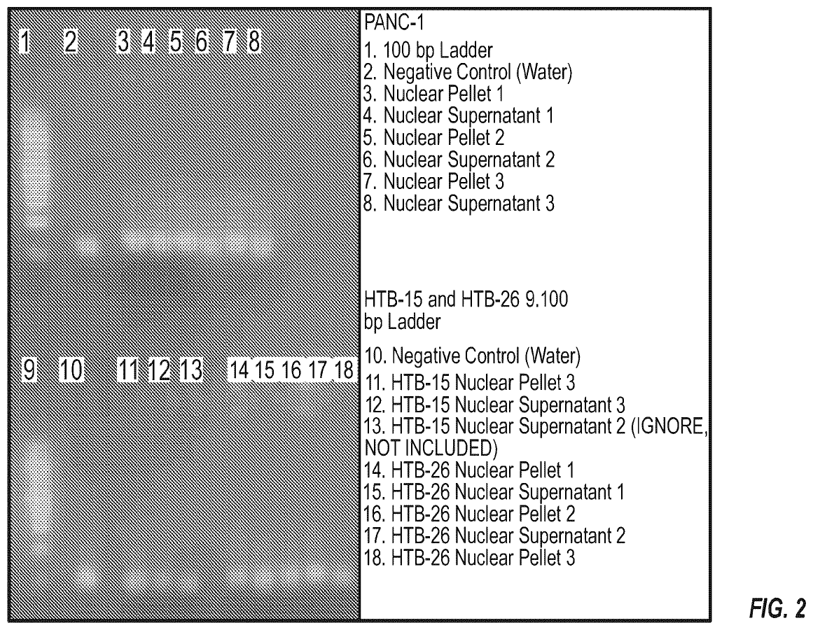

A random library of compounds related to the compound of the formula (X) was synthesized, each compound differing from the other by the aptamers that can be located between the two phosphates. The compounds in the library were labeled with “cold” indium (i.e., non-radioactive indium). The resulting indium chelates were exposed to three separate cell lines, namely, HTB-15 (glioblastoma); PANC-1 (pancreatic cancer); and HTB-26 (triple negative breast cancer). A nucleus isolation technique on each of the cell lines exposed to the indium chelates yielded a supernatant containing the nucleus membrane and a nucleus pellet. FIG. 2 is a photograph of a gel showing the successful isolation of drug candidates. The compounds isolated from the nucleus of the cells tested were submitted for next generation sequencing using Perkin Elmer's NGS Express platform using a forward primer:

forward primer:
(SEQ ID NO: 127)
TCGTCGGCAGCGTCAGATGTGTATAAGAGACAG-
[locus-specific];
reverse primer:
(SEQ ID NO: 128)
GTCTCGTGGGCTCGGAGATGTGTATAAGAGACAG-
[locus-specific];
forward indexing primer:
(SEQ ID NO: 129)
AATGATACGGCGACCACCGAGATCTACAC[i5]TCGTCGGCAGCGTC;
and
reverse indexing primer:
(SEQ ID NO: 130)
CAAGCAGAAGACGGCATACGAGAT[i7]GTCTCGTGGGCTCGG;

wherein [i5] and [i7] refer to the index sequence codes used by Illumina.

The adapter sequences (for post-run trimming) included: Read 1:

(SEQ ID NO: 131)
CTGTCTCTTATACACATCTCCGAGCCCACGAGACNNNNNNNNATCTCGTA
TGCCGTCTTCTGCTTG

### Read 2:

(SEQ ID NO: 132)
CTGTCTCTTATACACATCTGACGCTGCCGACGANNNNNNNNGTGTAGATC
TCGGTGGTCGCCGTATCATT

Thorium and lutetium chelates have also been prepared. Those chelates, like the indium chelates, were shown to be easily prepared and stable at a pH of 6 or below.

### Example 4: Compounds

Compounds of the following formulae A-D can be synthesized by methods available to those skilled in the art:

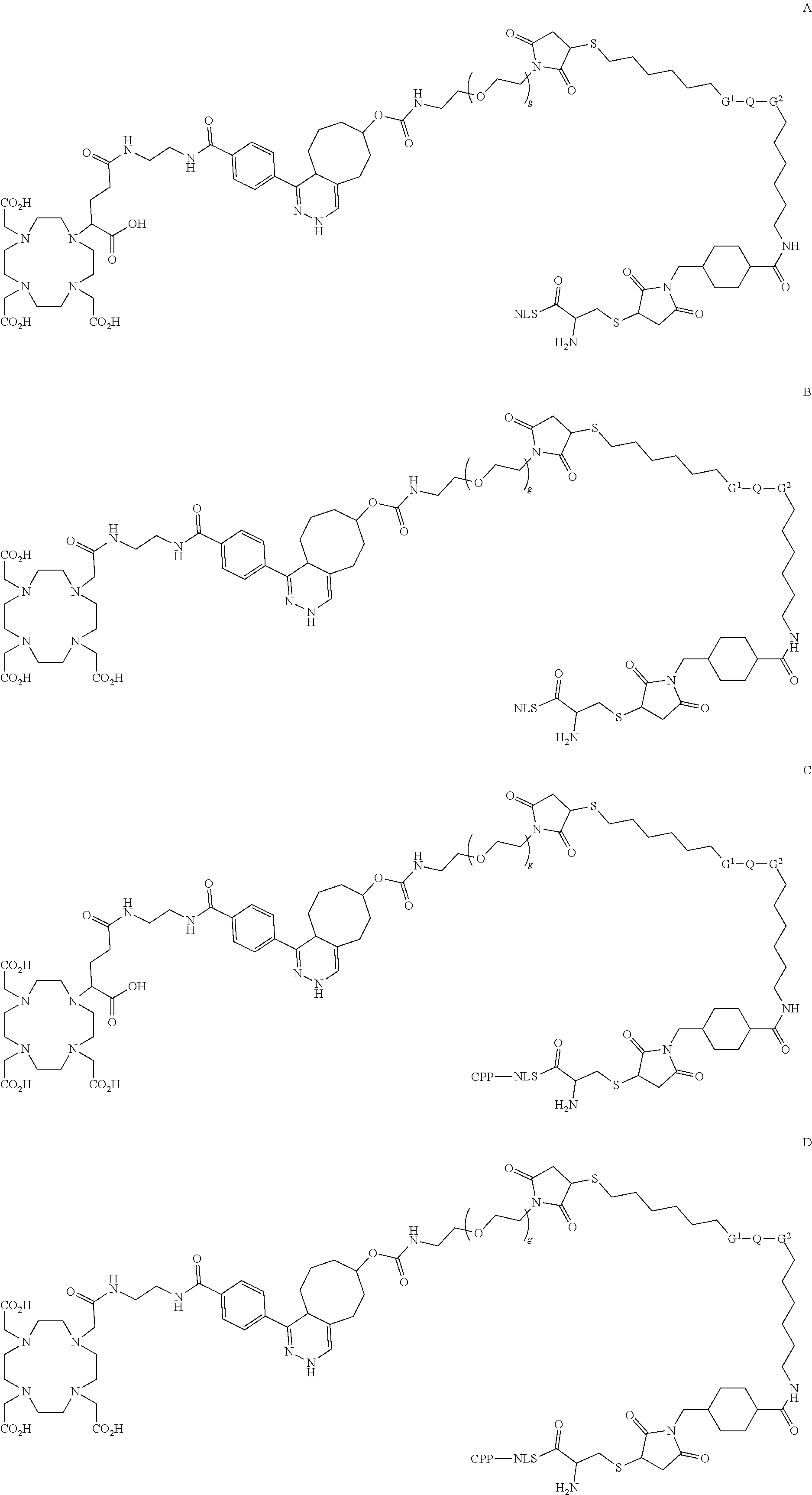

All of which can be synthesized from a compound of the formula:

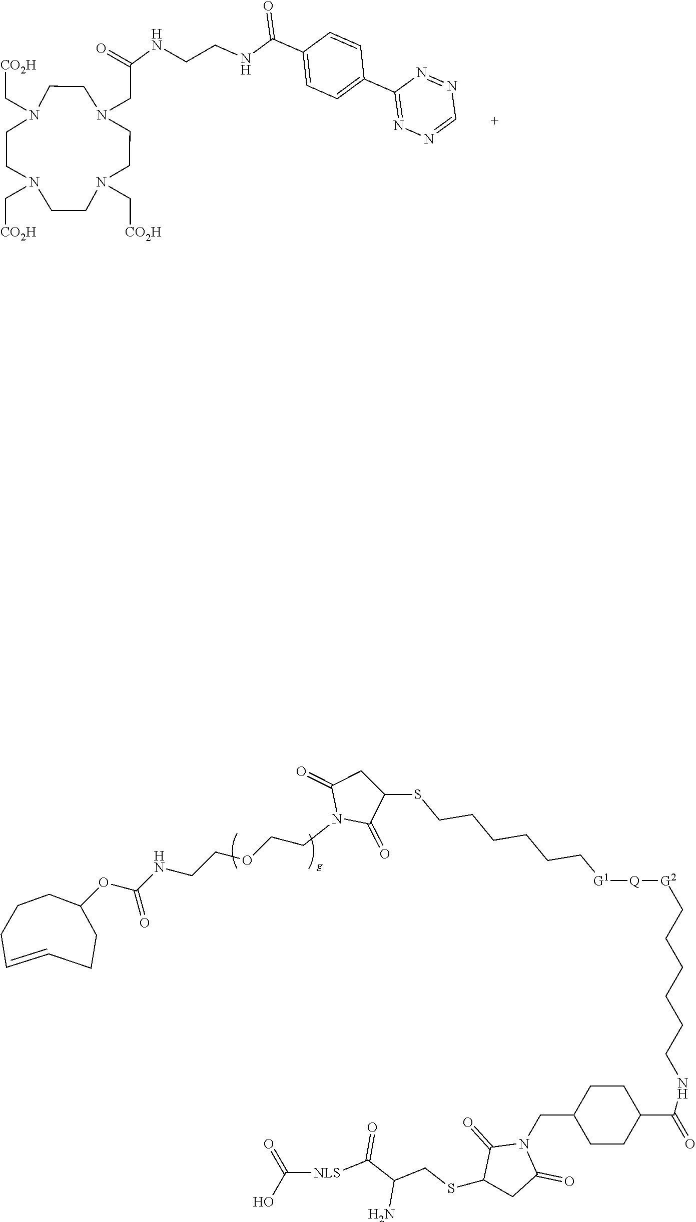

wherein G¹, G², Q, NLS, and CPP are defined herein and g is an integer such that the number average molecular weight (Mₙ) of the —OCH₂CH₂— moiety in the foregoing examples is from about 500 g/mol to about 10,000 g/mol (e.g., about 500 g/mol to about 5,000 g/mol, about 2,500 g/mol to about 7,500 g/mol, and about 5,000 g/mol to about 10,000 g/mol). An example of G¹, Q, G² is a single-stranded DNA sequence *TGCGTGTGTAGTGTGTCTG-(N*)25-CTCTTAGGGATTTGGGCGG* (SEQ ID NO:133). Alternatively, or in addition, NLS has the sequence PKKKRKV (SEQ ID NO:1) and can be incorporated into the foregoing compounds using a compound of the formula H₂N—PKKKRKV—CO₂H (SEQ ID NO:1).

### Example 5: In Vivo Approach

In this example, one or more compounds of the Formulae (I)-(X) is administered to a subject intravenously. Initially, the one or more compounds of the Formulae (I)-(X) are administered without, e.g., a therapeutic radionuclide “on board” (e.g., as a chelate) or with a non-toxic metal (e.g., non-radioactive isotope; in the case of indium, a non-radioactive isotope would be ¹¹³In and a radioactive isotope is ¹¹¹In). Examples of therapeutic radionuclides include, but are not limited to, alpha particle emitters (e.g., ²¹¹At, ²¹³Bi, ²²³Ra, ²²⁷Th and ²²⁵Ac), beta particle emitters (e.g., ³³P, ¹⁷⁷Lu, ⁶⁷Cu, ¹³¹I, ¹⁸⁶Re, ¹⁶⁵Dy, ⁸⁹Sr, ³²P, ¹⁶⁶Ho, ¹⁸⁸Re, and ⁹⁰Y), and Auger-electron emitters (e.g., ¹²⁵I, ¹²³I, ⁷⁷Br, ¹¹¹In, and ^{195m}Pt). See, Semin. Nucl. Med. 38:358-366 (2008). Some, but not all of the molecules of the Formulae (I)-(VIII) will bind to the cell membrane or be able to permeate through the cell membrane of tumor cells and into the cytoplasm because, for example, they have an appropriate CPP. Then, some, but not all of the molecules of the Formulae (I)-(IV) will be able to localize in the nucleus of the tumor tissue because, for example, they have an appropriate NLS in addition to or instead of a CPP. The tumor tissue can then be biopsied and one can isolate molecules of the Formulae (I)-(VIII) from the cell membrane and those found in the nucleus and the cytoplasm. The isolated molecules can then be sequenced to identify, among other things, the sequence of Q. The molecules that localized on the cell membrane or in the cytoplasm or in the nucleus can then be synthesized and radioactive versions of those molecules can be made for treatment, such as systemic or localized treatment.

### Example 6: Ex Vivo Approach

In this example, biopsied tumor tissue and “normal” tissue are treated with one or more compounds of the Formulae (I)-(X) ex vivo. The tumor tissue and the normal tissue are each treated with a “version” of the one or more compounds of the Formulae (I)-(X) without, e.g., a therapeutic radionuclide “on board” (e.g., as a chelate) or with a non-toxic metal (e.g., non-radioactive isotope; in the case of indium, the non-radioactive isotope would be ¹¹³In and the radioactive isotope is ¹¹¹In). Some, but not all of the molecules of the Formulae (I)-(X) will bind to the cell membrane or be able to permeate through the cell membrane of tumor cells and into the cytoplasm because, for example, they have an appropriate CPP. The same will be true for the normal tissue. Then, some, but not all of the molecules of the Formulae (I)-(X) will be able to localize on the cell membrane, in the cytoplasm or in the nucleus of the tumor tissue because, for example, they have an appropriate NLS in addition to or instead of a CPP. The same will be true for the normal tissue. One can then isolate molecules of the Formulae (I)-(X) found on or in, e.g., the cell membrane, the nucleus and the cytoplasm of the tumor tissue and the normal tissue. The isolated molecules can then be sequenced to identify, among other things, the sequence of Q. The molecules that localized on the cell membrane or in the cytoplasm or in the nucleus can then be synthesized and radioactive versions of those molecules can be made for treatment, such as systemic treatment or localized treatment.

Once isolated molecules from the tumor tissue are sequenced, they will be compared to the isolated molecules from normal tissue. Matching sequences from tumor tissue and normal tissue will be “subtracted” such that one can identify tumor specific molecules/compounds.

Select embodiments of the present disclosure include, but are not limited to, the following:

Embodiment 1 relates to a compound of the Formula I:

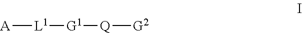

wherein
A comprises at least one avidin-type molecule, each avidin-type molecule comprising one to four monomer units;
L¹ is a linker group;
G¹ is a forward primer binding site;
Q is a randomized single-stranded DNA; and
G² is a reverse primer binding site.

Embodiment 2 relates to a compound of the Formula (Ia):

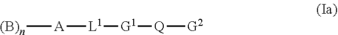

wherein B represents a biotin molecule or an analog thereof and n is an integer from 1 to 4, such that there can be at least one, at least two, at least three or four biotins attached to an avidin-type molecule.

Embodiment 3 relates to the compound of Embodiments 1-2, wherein the avidin-type molecule comprises four monomer units.

Embodiment 4 relates to the compound of Embodiments 1-2, wherein the avidin-type molecule is avidin, streptavidin, neutravidin or captavidin.

Embodiment 5 relates to the compound of Embodiments 1-4, wherein the randomized single-stranded DNA has 10 to 100 nucleotides.

Embodiment 6 relates to the compound of Embodiments 1-5, wherein L¹ is a click chemistry-derived linker.

Embodiment 7 relates to the compound of Embodiments 1-6, wherein the linker is derived from copper-catalyzed azide-alkyne cycloaddition (CuAAC), strainpromoted azide-alkyne cycloaddition (SPAAC), inverse electron demand Diels-Alder reaction (IEDDA), and Staudinger ligation (SL).

Embodiment 8 relates to the compound of Embodiments 1-7, wherein the linker group is a releasable group.

Embodiment 9 relates to the compound of Embodiments 1-8, wherein the linker group is photochemically-, chemically- or enzymatically-cleavable group.

Embodiment 10 relates to the compound of Embodiments 1-9, wherein at least one of G¹, Q, and G² comprises at least one bonding arrangement that renders at least one of G¹, Q, and G² nuclease resistant.

Embodiment 11 relates to the compound of Embodiment 10, wherein the at least one bonding arrangement is a 5′-phosphorothioate, 5-modified uracil, 4′-thio, 2′-fluoro, 5′-α-P-borano, 2′-amino, 2′-deoxy-L-ribose, 2′-methoxy, capping with 3′ end with inverted thymidine, bridged nucleic acids (BNAs), locked nucleic acids (LNAs), and xeno nucleic acids (XNAs).

Embodiment 12 relates to the compound of Embodiments 1-11, further comprising a nuclear localization signal (NLS), such that the compound of the Formula I is a compound of the Formula II:

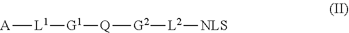

Embodiment 13 relates to the compound of Embodiments 12, further comprising a cell penetrating peptide (CPP) such that the compound of the Formula I is a compound of the Formula (III):

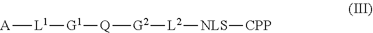

Embodiment 14 relates to the compound of Embodiments 12-13, further comprising a linker, L³, linking NLS to CPP.

Embodiment 15 relates to the compound of Embodiments 12-14, wherein at least one of L² and L³ is a succinimidyl 4-(N-maleimidomethyl)cyclohexane-1-carboxylate-based linker.

Embodiment 16 relates to the compound of Embodiments 12-15, wherein the NLS comprises the amino acid sequence PKKKRKV (SEQ ID NO:1).

Embodiment 17 relates to the compound of Embodiments 12-16, wherein the CPP comprises the amino acid sequence RRRRRRRR (SEQ ID NO:5).

Embodiment 18 relates to the compound of Embodiments 1-17, further comprising at least one biotin, or analog thereof, bound to at least one of the avidin-type molecule.

Embodiment 19 relates to the compound of Embodiment 17, wherein the at least one derivative of biotin is modified so that it is biotinidase resistant.

Embodiment 20 relates to the compound of Embodiment 18, wherein the at least one biotin comprises at least one chelating group attached to the at least one biotin.

Embodiment 21 relates to the compound of Embodiments 20, wherein the chelating agent is EDTA, DTPA, DOTA, TETA, NOTA, Cyclam, PCBA, DADT or MAMA.

Embodiment 22 relates to the compound of Embodiments 20-21, further comprising at least one of a radioactive isotope and a non-radioactive isotope chelated to the chelating agent.

Embodiment 23 relates to the compound of Embodiment 22, wherein the radioactive isotope is at least one of an alpha particle emitter, a beta particle emitter, and an Auger-electron emitter.

Embodiment 24 relates to the compound of Embodiment 23, wherein the non-radioactive isotope is ¹¹³In.

Embodiment 25 relates to the compound of Embodiment 23, wherein the alpha particle emitter is at least one of ²¹¹At, ²¹³Bi, ²²³Ra, ²²⁷Th and ²²⁵Ac.

Embodiment 26 relates to the compound of Embodiment 23, wherein the beta particle emitter is at least one of ³³P, ¹⁷⁷Lu, ⁶⁷Cu, ¹³¹I, ¹⁸⁶Re, ¹⁶⁵Dy, ⁸⁹Sr, ³²P, ¹⁶⁶Ho, ¹⁸⁸Re, and ⁹⁰Y.

Embodiment 27 relates to the compound of Embodiment 23, wherein the Auger-electron emitter is at least one of ¹²⁵I, ¹²³I, ⁷⁷Br, ¹¹¹In, and ^{195m}Pt.

Embodiment 28 relates to the compound of Embodiments 1-27, wherein -G¹-Q-G²- is a single-stranded DNA sequence -TGCGTGTGTAGTGTGTCTG-Q-CTCTTAGGGATTTGGGCGG- (SEQ ID NO:21), wherein optionally comprises at least one bonding arrangement that renders at least one of G¹, Q, and G² nuclease resistant.

Embodiment 29 relates to the compound of Embodiments 1-27, wherein at least one of G¹, Q, and G² comprises at least one bonding arrangement that renders at least one of G¹, Q, and G² nuclease resistant.

Embodiment 30 relates to the compound of Embodiment 29, wherein the at least one bonding arrangement is a 5′-phosphorothioate, 5-modified uracil, 4′-thio, 2′-fluoro, 5′-α-P-borano, 2′-amino, 2′-deoxy-L-ribose, 2′-methoxy, capping with 3′ end with inverted thymidine, bridged nucleic acids (BNAs), locked nucleic acids (LNAs), and xeno nucleic acids (XNAs).

Embodiment 31 relates to a pharmaceutical composition comprising one or more compounds of Embodiments 1-30 and a pharmaceutically acceptable carrier or excipient.

Embodiment 32 relates to a method of treating cancer in a subject in need of such treatment, comprising administering a therapeutically-effective amount of one or more compounds of Embodiments 1-30.

Embodiment 33 relates the method of Embodiment 32, wherein the cancer is selected from all possible cancers, including prostate cancer, breast cancer, pancreatic cancer, thyroid cancer, bone cancer, glioblastoma and neuroendocrine tumors.

Embodiment 34 relates to the method of Embodiment 32-33, further comprising administering one or more of compounds of Embodiments 1-30 in combination with at least one anticancer agent.

Embodiment 35 relates to a compound of the Formula (V):

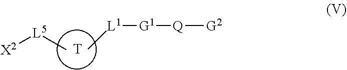

or a pharmaceutically acceptable salt, polymorph, prodrug, solvate or clathrate thereof, wherein:
X² is a chelating agent, an imaging agent, a diagnostic agent or a therapeutic agent;
L¹ is a linker group;
L⁵ is a linker group or a bond, wherein L¹ and L⁵ can be the same or different;
T is a click-chemistry-derived core;
G¹ is a forward primer binding site;
Q is a randomized single-stranded DNA; and
G² is a reverse primer binding site.

Embodiment ³⁶ relates to the compound of Embodiment ^{35,} wherein X² is a chelating agent.

Embodiment ³⁷ relates to the compound of Embodiments ³⁵-^{36,} wherein the chelating agent is EDTA, DTPA, DOTA, TETA, NOTA, Cyclam, PCBA, DADT or MAMA.

Embodiment ³⁸ relates to the compound of Embodiments ³⁶-^{37,} further comprising at least one of a radioactive isotope and a non-radioactive isotope chelated to the chelating agent.

Embodiment 39 relates to the compound of Embodiment 38, wherein the radioactive isotope is at least one of an alpha particle emitter, a beta particle emitter, and an Auger-electron emitter.

Embodiment 40 relates to the compound of Embodiment 38, wherein the non-radioactive isotope is ¹¹³In.

Embodiment 41 relates to the compound of Embodiment 38, wherein the alpha particle emitter is at least one of ²¹¹At, ²¹³Bi, ²²³Ra, ²²⁷Th and ²²⁵Ac.

Embodiment 42 relates to the compound of Embodiment 38, wherein the beta particle emitter is at least one of ³³P, ¹⁷⁷Lu, ⁶⁷Cu, ^{1311, 186}Re, ¹⁶⁵Dy, ⁸⁹Sr, ³²P, ¹⁶⁶Ho, ¹⁸⁸Re, and ⁹⁰Y.

Embodiment 39 relates to the compound of Embodiment 38, wherein the Auger-electron emitter is at least one of ¹²⁵I, ¹²³I, ⁷⁷Br, ¹¹¹In, and ^{195m}Pt.

Embodiment 40 relates to the compound of Embodiments 35-39, wherein the randomized single-stranded DNA has 10 to 100 nucleotides.

Embodiment 41 relates to the compound of Embodiments 35-40, wherein -G¹-Q-G²- is a single-stranded DNA sequence -TGCGTGTGTAGTGTGTCTG-Q-CTCTTAGGGATTTGGGCGG- (SEQ ID NO:21), wherein optionally comprises at least one bonding arrangement that renders at least one of G¹, Q, and G² nuclease resistant.

Embodiment 42 relates to the compound of Embodiments 35-41, wherein at least one of L¹ and L⁵ is a click chemistry-derived linker.

Embodiment 43 relates to the compound of Embodiments 35-42, wherein at least one of L¹ and L⁵ is derived from copper-catalyzed azide-alkyne cycloaddition (CuAAC), strainpromoted azide-alkyne cycloaddition (SPAAC), inverse electron demand Diels-Alder reaction (IEDDA), and Staudinger ligation (SL).

Embodiment 44 relates to the compound of Embodiments 35-43, wherein at least one of L¹ and L⁵ is a releasable group.

Embodiment 45 relates to the compound of Embodiment 35-44, wherein at least one of L¹ and L⁵ is photochemically-, chemically- or enzymatically-cleavable group.

Embodiment 46 relates to the compound of Embodiments 35-45, wherein at least one of G¹, Q, and G² comprises at least one bonding arrangement that renders at least one of G¹, Q, and G² nuclease resistant.

Embodiment 47 relates to the compound of Embodiment 46, wherein the at least one bonding arrangement is a 5′-phosphorothioate, 5-modified uracil, 4′-thio, 2′-fluoro, 5′-α-P-borano, 2′-amino, 2′-deoxy-L-ribose, 2′-methoxy, capping with 3′ end with inverted thymidine, bridged nucleic acids (BNAs), locked nucleic acids (LNAs), and xeno nucleic acids (XNAs).

Embodiment 48 relates to a compound of Embodiments 35-47, further comprising a nuclear localization signal (NLS), such that the compound of the Formula (V) is a compound of the Formula (VI):

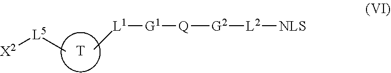

Embodiment 49 relates to the compound of Embodiments 35-48, further comprising a nuclear localization signal (NLS) and further comprising a cell penetrating peptide (CPP) such that the compound of the Formula (V) is a compound of the Formula (VII):

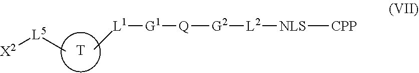

Embodiment 50 relates to the compound of Embodiment 49, further comprising a linker, L³, linking NLS to CPP.

Embodiment 51 relates to the compound of claim 50, wherein at least one of L² and L³ is a succinimidyl 4-(N-maleimidomethyl)cyclohexane-1-carboxylate-based linker.

Embodiment 52 relates to the compound of Embodiments 48-51, wherein the NLS comprises the amino acid sequence PKKKRKV (SEQ ID NO:1).

Embodiment 53 relates to the compound of Embodiments 35-47, further comprising a cell penetrating peptide (CPP) such that the compound of the Formula (V) is a compound of the Formula (VIII):

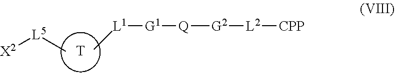

Embodiment 54 relates to the compound of Embodiments 49-53, wherein the CPP comprises the amino acid sequence RRRRRRRR (SEQ ID NO:5).

Embodiment 55 relates to the compound of Embodiments 35-47, wherein the compound is a compound of the formula:

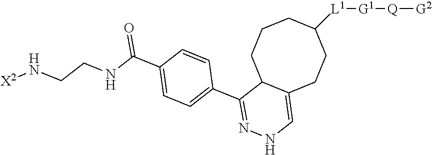

or a pharmaceutically acceptable salt, polymorph, prodrug, solvate or clathrate thereof, wherein.

Embodiment 56 relates to the compound of Embodiment 55, wherein the compound is a compound of the formula:

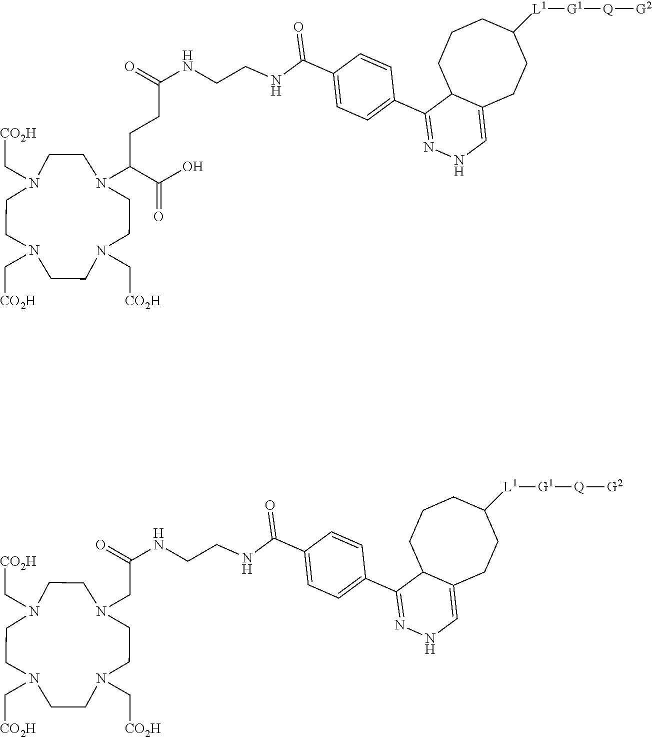

or a pharmaceutically acceptable salt, polymorph, prodrug, solvate or clathrate thereof.

Embodiment 57 relates to the compound of Embodiments 35-47, wherein the compound is a compound of the formula:

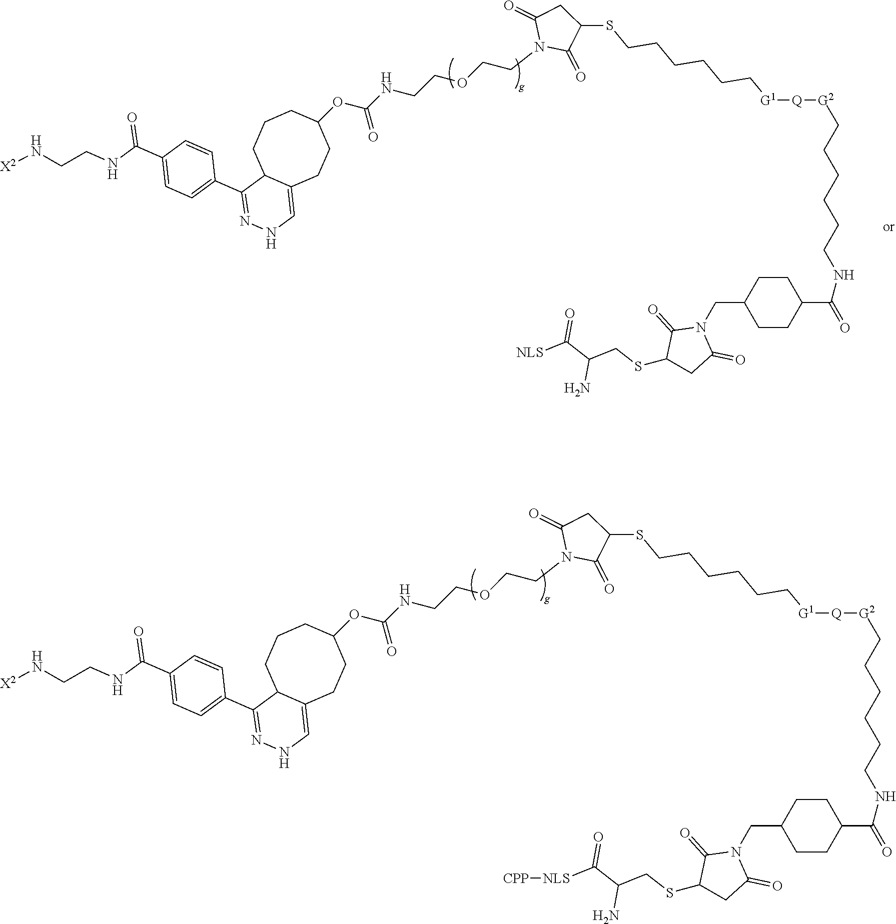

wherein g is an integer such that the number average molecular weight (Mn) of the —OCH₂CH₂— moiety is from about 500 g/mol to about 10,000 g/mol.

Embodiment 58 relates to a pharmaceutical composition comprising one or more compounds of Embodiments 35-57 and a pharmaceutically acceptable carrier or excipient.

Embodiment 59 relates to a method of treating cancer in a subject in need of such treatment, comprising administering a therapeutically-effective amount of one or more compounds of Embodiments 35-57.

Embodiment 59 relates to the method of Embodiment 59, wherein the cancer is selected from all possible cancers, including prostate cancer, breast cancer, pancreatic cancer, thyroid cancer, bone cancer, glioblastoma and neuroendocrine tumors.

Embodiment 60 relates to the method of Embodiments 59-60, further comprising administering one or more of compounds of Embodiments 35-57 in combination with at least one anticancer agent.

What is claimed is:

1. A compound of the Formula (V): 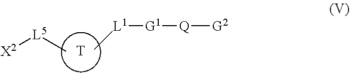
or a pharmaceutically acceptable salt thereof,
wherein:
X² is a chelating agent-selected from EDTA, DTPA, DOTA, TETA, NOTA, Cyclam, PCBA, DADT, and MAMA;
L¹ is a linker group;
L⁵ is a linker group or a bond, wherein L¹ and L⁵ can be the same or different, wherein L¹ and/or L⁵ has a chain length of from 1 to 40 atoms;
T is a click-chemistry-derived core;
G¹ is a single-stranded nucleotide sequence for binding a forward primer;
Q is a randomized single-stranded DNA;
G² is a single-stranded nucleotide sequence for binding a reverse primer wherein the click-chemistry-derived core is (i) a triazole, (ii) an oxazole, (iii) a heterocycle derivable from electrocyclization chemistry, or (iv) selected from one of the following formula: 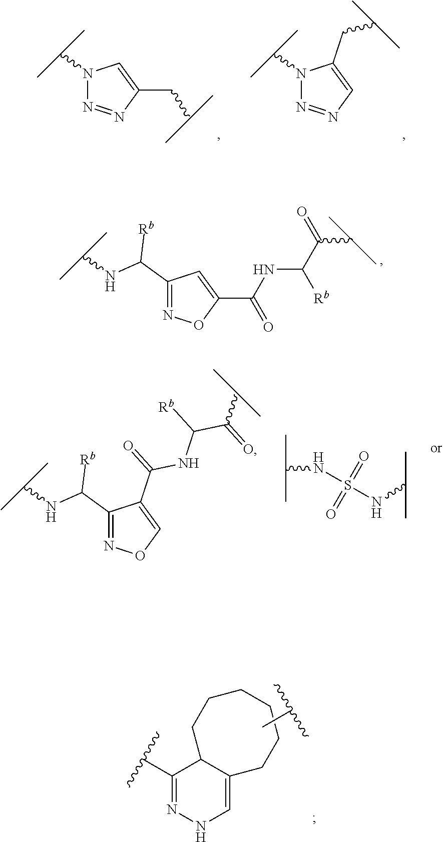
wherein R^{b} is H, alkyl, arylalkyl, -alkyl-S-alkyl or arylalkyl or the side-chain of any naturally- or non-naturally occurring amino acid; and
wherein the compound of the formula (V) is internalized into at least one of the cytoplasm and the nucleus of cancer cells.

2. The compound of claim 1, wherein the chelating agent is DTPA, DOTA, TETA, or NOTA.

3. The compound of claim 1, further comprising at least one of a radioactive isotope and a non-radioactive isotope chelated to the chelating agent.

4. The compound of claim 1, wherein the randomized single-stranded DNA has 10 to 100 nucleotides.

5. The compound of claim 1, wherein -G¹-Q-G²- is a single-stranded DNA sequence -TGCGTGTGTAGTGTGTCTG-Q-CTCTTAGGGATTTGGGGGG- (SEQ ID NO:21), wherein optionally comprises at least one bonding arrangement that renders at least one of G¹, Q, and G² nuclease resistant.

6. The compound of claim 1, wherein at least one of L¹ and L⁵ is (i) a click chemistry-derived linker, wherein at least one of L¹ and L⁵ is derivable from copper-catalyzed azide-alkyne cycloaddition (CuAAC), strain-promoted azide-alkyne cycloaddition (SPAAC), inverse electron demand Diels-Alder reaction, (IEDDA), and Staudinger ligation (SL); or (ii) a releasable group, optionally wherein the linker group is photochemically-, chemically- or enzymatically cleavable group.

7. The compound of claim 1, wherein at least one of L¹ and L⁵ is a releasable group.

8. The compound of claims 1, wherein at least one of G¹, Q, and G² comprises at least one bonding arrangement that renders at least one of G¹, Q, and G² nuclease resistant.

9. The compound of claim 1, further comprising a at least one of a cell penetrating peptide (CPP) and a nuclear localization sequence (NLS).

10. The compound of claim 1, wherein the NLS comprises the amino acid sequence PKKKRKV (SEQ ID NO:1).

11. The compound of claim 9, wherein the CPP comprises the amino acid sequence RRRRRRRR (SEQ ID NO:5).

12. The compound of claim 1, wherein the compound is a compound of the formula: 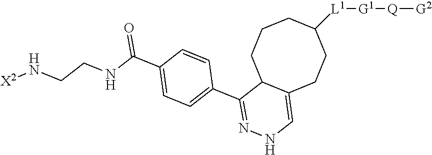
or a pharmaceutically acceptable salt thereof, wherein.

13. The compound of claim 12, wherein the compound is a compound of the formula: 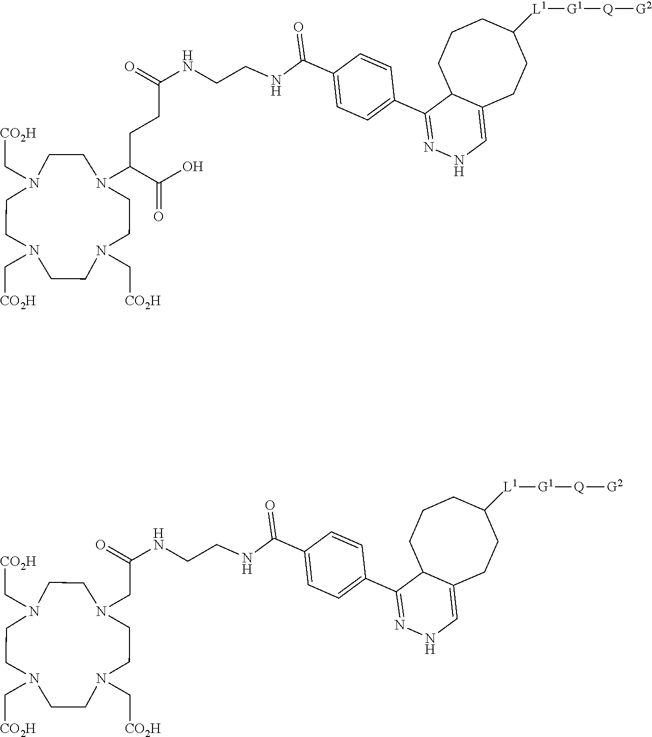
or a pharmaceutically acceptable salt thereof.

14. The compound of claim 1, wherein the compound is a compound of the formula: 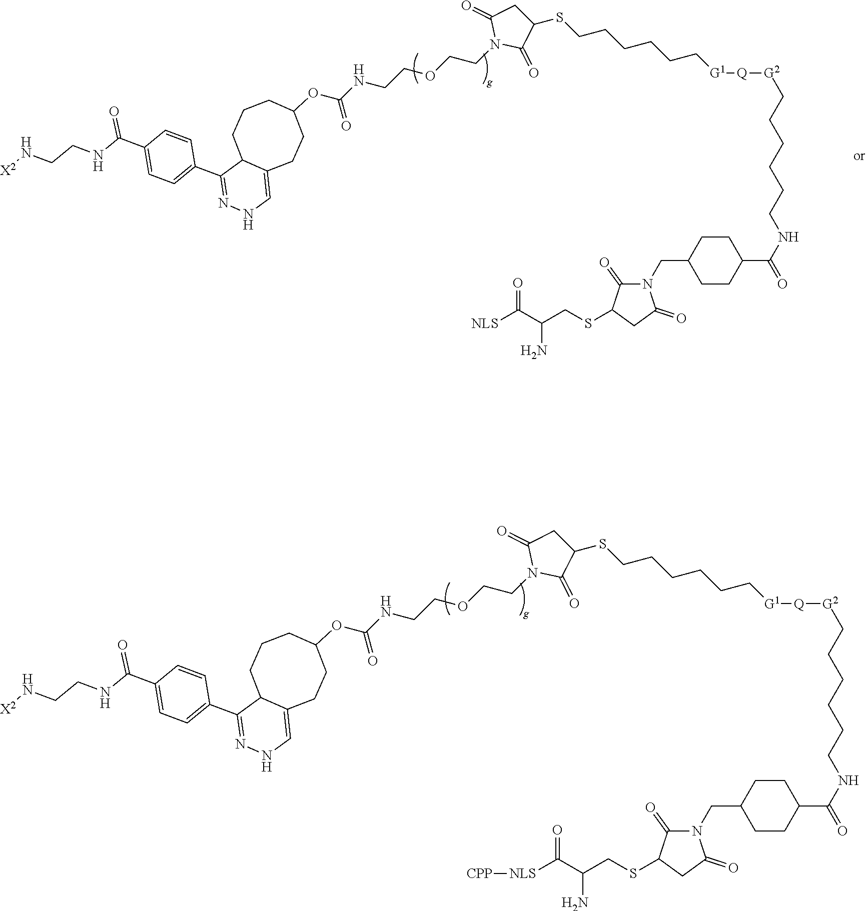
or a pharmaceutically acceptable salt thereof;
wherein g is an integer such that the number average molecular weight (Mn) of the —OCH₂CH₂— moiety is from about 500 g/mol to about 10,000 g/mol.

15. A pharmaceutical composition comprising one or more compounds of claim 1 and a pharmaceutically acceptable carrier or excipient.

16. A method of treating cancer in a subject in need of such treatment, comprising administering a therapeutically-effective amount of one or more compounds of claim 1.

17. The method of claim 16, wherein the cancer is selected from prostate cancer, breast cancer, pancreatic cancer, thyroid cancer, bone cancer, glioblastoma, and neuroendocrine tumors.

18. The method of claim 16, further comprising administering one or more of compounds of claim 1 in combination with at least one anticancer agent.

19. The compound of claim 1, wherein the chelating agent is DOTA.

20. The compound of claim 1, wherein Q comprises the sequence:
(SEQ ID NO: 22)
TCGGGATCAACCCGAGTTCACGCAACT;
(SEQ ID NO: 23)
ACACTAGTACAGTCAGTACGCACGA;
(SEQ ID NO: 24)
TTTTGCGCCTGAAGCCTCCCCAGGA;
(SEQ ID NO: 25)
GTCCGTATCTTGGTCGAAGATGTAC;
(SEQ ID NO: 26)
AGGTTCAATCTACCTTCTGCCATGC;
(SEQ ID NO: 27)
CTGGAATTCCAAATATGCCGGCGAG;
(SEQ ID NO: 28)
ATCGTTCTGGGATACAAGCTTTTGA;
(SEQ ID NO: 29)
AAATATACATTATTCCCATCAAAAT;
(SEQ ID NO: 30)
GGACAAAAAGTCTGAGTCTGACCTT;
(SEQ ID NO: 31)
GATGGCAGTATAGTCGTCATGAGTC;
(SEQ ID NO: 32)
TCGGGATCAACCCGAGTTCACGCAA;
(SEQ ID NO: 33)
ACTCTTTCTGTCACAAGATCTGCAT;
(SEQ ID NO: 34)
AATGTGGATGGCATAGTGGCGGCGC;
(SEQ ID NO: 35)
TACAACCTAGAGGATGAGCTCACGA;
(SEQ ID NO: 36)
TTAGTGTTACATCACATCTCGAGCT;
(SEQ ID NO: 37)
TAGGAGGCATTCATTTTTAAGGTAG;
(SEQ ID NO: 38)
GTTACCACATCGATCTGCGAAAACT;
(SEQ ID NO: 39)
CACCAGTGGTTTGATATACGGCCTA;
(SEQ ID NO: 40)
TAGGTTGTGGTTCCCGAATCGTGAG;
(SEQ ID NO: 41)
GCAACTTGGCTTCCGTCTAAACAAA;
(SEQ ID NO: 42)
GCTATCCTTCTTTTAGCAGACAGTA;
(SEQ ID NO: 43)
TACTCGTCGCGTTTTATTTTTTTGC;
(SEQ ID NO: 44)
ATAAGCCCCCAGCTACTCCCGTTTT;
(SEQ ID NO: 45)
CTTCATGTAGCAACTCATTGTGAAG;
(SEQ ID NO: 46)
TCCCAAAATCTCGCCCCCCGGAATA;
(SEQ ID NO: 47)
AGTTGCCACAAAAATTTACTAAGTC;
(SEQ ID NO: 48)
CAGCTACATTCATTATTTGTTTCCG;
(SEQ ID NO: 49)
TCATACGGAACCTCAGCCCATGACA;
(SEQ ID NO: 50)
CACTTATTAGAAATGCATACCTATA;
(SEQ ID NO: 51)
GTCTGAAATTAATTGATCGTCATGC;
(SEQ ID NO: 52)
GCATCCGATTCACACACTCGCTCAC;
(SEQ ID NO: 53)
CATAACAGATGTTAAATTAGCGTAA;
(SEQ ID NO: 54)
AAAATCATGAGTGGTTTACCGGTAG;
(SEQ ID NO: 55)
CAACCCGCAAAAAGTCTCAGGAGTA;
(SEQ ID NO: 56)
CAGCTTCGCCATCCCTACGGGTAAG;
(SEQ ID NO: 57)
TTCTGCATGGCGGGTATACTCACTA;
(SEQ ID NO: 58)
CCCGTAGCCAAGGAGCCTATACAAC;
(SEQ ID NO: 59)
TTTAAGTTTCCGAATCCAACGTAAA;
(SEQ ID NO: 60)
GGGGAGCAGCAGCGATTTGACCTAG;
(SEQ ID NO: 61)
GAACCAAAGCGCTGCTTCCCATAAA;
(SEQ ID NO: 62)
ATGGTTATCTTACCCTACCAAAGGA;
(SEQ ID NO: 63)
GACCGGGTTCATTTAACCGTACGGG;
(SEQ ID NO: 64)
CTCCAACGCAGTAGCCAGGTACACA;
(SEQ ID NO: 65)
TAACTGTTGCCTCTCACATGGTCAA;
(SEQ ID NO: 66)
CGCTTAAGTGGTATAGTCTCCATCG;
(SEQ ID NO: 67)
TCGCACCCTATCATAGTCCGACACC;
(SEQ ID NO: 68)
GAATCTAAACGTTAGCAATCGGCGT;
(SEQ ID NO: 69)
CGGTTCCAGGTGGGCCCGTATACGT;
(SEQ ID NO: 70)
AGCTCCCCCCCGCGTTATACCTGAC;
(SEQ ID NO: 71)
GCCTAGCATCCAATCGACGTACCGC;
(SEQ ID NO: 72)
CACAACTTCCATATGTCAGTTCAAC;
(SEQ ID NO: 73)
AAGCAGTCTCATTCATCCATCACTA;
(SEQ ID NO: 74)
GCTTGGATTTAAACCAAGCGTCCCG;
(SEQ ID NO: 75)
TAGATATCGTATAATATGGGGATAA;
(SEQ ID NO: 76)
TAATGCCACACGTTATGTCTCCCAA;
(SEQ ID NO: 77)
ACGAAGGCGGCATGGTAATCTGCAC;
(SEQ ID NO: 78)
GGCGTGGGTTGACCGGATACATGAA;
(SEQ ID NO: 79)
TACAAGACCGAACCTGGTTTATACC;
(SEQ ID NO: 80)
GTGGACTCGACATCCGACAGTCAGT;
(SEQ ID NO: 81)
TAACCGGTTGGATAGCGATTCGATT;
(SEQ ID NO: 82)
GCCAGTCTAACAGTAATTATGCAAA;
(SEQ ID NO: 83)
TATTCATAATACATGAGATGGCACG;
(SEQ ID NO: 84)
CATAGGATTCGTAATGTATAAGTGT;
(SEQ ID NO: 85)
TATCTCAGAATACCGCACTCACGTG;
(SEQ ID NO: 86)
CGGTCAAAGCACCTGGAGCGTATTC;
(SEQ ID NO: 87)
CTGTACTTGATCCAAGGTTTGAAGG;
(SEQ ID NO: 88)
GACTACGTCGCTTGCAAATCATCCG;
(SEQ ID NO: 89)
TGTAGTCTATAACTCCCTGGCGCAG;
(SEQ ID NO: 90)
GGTAGGCAGCACGTTTGTGTGAACC;
(SEQ ID NO: 91)
CTCTTCACCATTTTATCGCCATGCA;
(SEQ ID NO: 92)
GTCTGTCTGTATCATCCGAGCGACA;
(SEQ ID NO: 93)
GGAAGCGGGATATATGGTGCCGTCC;
(SEQ ID NO: 94)
TAACTCAGACAGCTAGCTATCGTTA;
(SEQ ID NO: 95)
TAACTCTGCATTGCTCTCAGGGAGC;
(SEQ ID NO: 96)
GTGTCTTGTACTCTGACCTGAAGCG;
(SEQ ID NO: 97)
GGAATAAGAAGTCTTAGTAGCCCAC;
(SEQ ID NO: 98)
AAATAGTAAATTGAGGAGCCGTTTA;
(SEQ ID NO: 99)
CTACGTATAAACGGTTGGTTAGGTT;
(SEQ ID NO: 100)
AGCATGAATGGAGGCCGTTAACAAA;
(SEQ ID NO: 101)
TCCCGCAGTTATCGCGGCTGTCTCA;
(SEQ ID NO: 102)
CGCCGTAGGGCGTATGGCGCGTCTG;
(SEQ ID NO: 103)
CTACGTGCCAGTTTATACCCCGGAA;
(SEQ ID NO: 104)
CCGCTAGAGAACCTTGATGATTCTG;
(SEQ ID NO: 105)
TATCTTAAGTCAGTGGGGCTCGTCG;
(SEQ ID NO: 106)
TTTATGAAGAGCACATCATAAGAAG;
(SEQ ID NO: 107)
TGGCCGCCTAGAGTTAAGAACTATT;
(SEQ ID NO: 108)
GGGTCGAATCTAGTTTTGTAACAGG;
(SEQ ID NO: 109)
TGACAGTGGCGTCACCCGTTCACCC;
(SEQ ID NO: 110)
CTGCGTACTGGATATGTAAAAGATG;
(SEQ ID NO: 111)
GGAATGCGTGCAGACCCGTTGGTTT;
(SEQ ID NO: 112)
GTACCCAAATGTGAGTGACGCCATT;
(SEQ ID NO: 113)
CATCTCTGTGTACGGAAATCTTTGA;
(SEQ ID NO: 114)
GTTTAGCGATCCTTTTGAGCATTAG;
(SEQ ID NO: 115)
CTAAGTTGTAAGCGCAGCACAGCGT;
(SEQ ID NO: 116)
GAAACCTTTGTGCAACGCTCGTGTT;
(SEQ ID NO: 117)
CTTGTCTGTCCCGCTAGTTTGGGGG;
(SEQ ID NO: 118)
CTAAAACCGGGCGTAGACGATGGTC;
(SEQ ID NO: 119)
CGGTAGTCGCCGGCTTATATGCCGA;
(SEQ ID NO: 120)
TTCTGATATCTGATGTTTTATGGCT;
(SEQ ID NO: 121)
AGCTCGTCTAGAAACCGTGGGCCAA;
(SEQ ID NO: 122)
GATAAGGGTAGAGTCCAGTGAACGG;
or
wherein:
G¹-Q-G² comprises the sequence:
(SEQ ID NO: 123)
TGCGTGTGTAGTGTGTCTGTCGGGATCAACCCGAGTTCACGCAACTCTTA
GGGATTTGGGCGG;
(SEQ ID NO: 124)
TGCGTGTGTAGTGTGTCTGGACCGGGTTCATTTAACCGTACGGGCTCTTA
GGGATTTGGGCGG;
(SEQ ID NO: 125)
TGCGTGTGTAGTGTGTCTGGCTTGGATTTAAACCAAGCGTCCCGCTCTTA
GGGATTTGGGCGG;
or
(SEQ ID NO: 126)
TGCGTGTGTAGTGTGTCTGCGGTAGTCGCCGGCTTATATGCCGACTCTTA
GGGATTTGGGCGG.
